(19) United States
(12) Patent Application Publication
(10) Pub. No.: US 2024/0366086 A1
(43) Pub. Date: Nov. 7, 2024

(54) Contact Lens System and Method for Assessing Intraocular Pressure

(71) Applicant: The Regents of the University of California, Oakland, CA (US)

(72) Inventors: O'Rese Knight, Orinda, CA (US); Noël Ziebarth, Miami, FL (US); Noa H. Dehaseth, Miami, FL (US)

(21) Appl. No.: 18/655,182

(22) Filed: May 3, 2024

Related U.S. Application Data
(60) Provisional application No. 63/499,958, filed on May 3, 2023.
(60) Provisional application No. 63/607,865, filed on Dec. 8, 2023.

(51) Int. Cl. A61B 3/16; A61F 9/00

(52) U.S. Cl. CPC A61B 3/16; A61F 9/0017

(57) ABSTRACT

Devices, systems, and/or methods for assessing intra-ocular pressure of the cornea of an eye using a wearable sensor, and/or the use of such assessments to monitor, diagnose, and/or treat glaucoma in patients.

### RELATED APPLICATIONS

This application claims priority to U.S. Provisional Patent Application No. 63/499,958, filed May 3, 2023, and to U.S. Provisional Patent Application No. 63/607,865, filed Dec. 8, 2023, the contents of both of which are incorporated herein by reference in their respective entireties.

### FIELD

The various embodiments herein relate to devices, systems, and/or methods for assessing biomechanical properties of the cornea of an eye using a wearable sensor, and/or using such assessments to monitor, diagnose, and/or treat conditions such as glaucoma.

### BACKGROUND

Glaucoma is a serious and complex eye disease that can induce optic nerve damage and visual field loss. Glaucoma is the leading cause of irreversible vision loss or blindness worldwide and will affect nearly 5 million Americans and 111.8 million people worldwide by 2040. Glaucoma is generally linked to high intraocular pressure (“IOP”), which is the only modifiable risk factor. Accordingly, IOP is routinely measured in eye exams as a tool for the screening, diagnosis and management of glaucoma. The gold standard for measuring IOP is the Goldman applanation tonometer (GAT). This device makes the incorrect assumption that the cornea is a thin membrane. FIG. 1 is a side cross-sectional view of a portion of an eye of a patient, illustrating the relationship between external forces acting on the cornea of the eye and the corneal elasticity and intraocular pressure. Corneal elasticity may vary between patients and may confound IOP measurements acquired with applanation tonometry. Moreover, GAT and many of the other commercially available tonometers are only capable of acquiring a single IOP measurement, and this is typically performed during the eye exam. IOP is a physiologic property that fluctuates naturally throughout the day. IOP may, for example vary with a baseline circadian rhythm and/or in response to physical activity, recumbency, and the cardiac cycle. Therefore, a single measurement during an eye exam may fail to provide a complete picture of the health of the eyes. Further, known IOP measurement technologies can typically only be used in a clinical/treatment environment such as a doctor's office or hospital, etc., and thus cannot be used to monitor IOP overnight unless the patient is required to participate in a sleep study and/or stay in the clinical environment overnight.

Goldmann applanation tonometry (“GAT”) is the current gold standard technique used to measure and quantify IOP. GAT assumes the cornea is an infinitely thin, elastic membrane. This assumption gives rise to significant measurement error due to the natural variation of central corneal thickness (“CCT”) among patients. This, in turn, may impede efficient clinical management of glaucoma. For example, patients with thick corneas may be over-diagnosed and subjected to an unnecessary healthcare burden, while patients with thin corneas may be denied treatment due to underdiagnosis.

The use of Atomic Force Microscopy (“AFM”) is discussed in U.S. Published App. No. 2023/0070316, the contents of which are incorporated by reference herein. FIG. 3 is a conceptual system diagram showing the use of (AFM) to measure elasticity, using a cantilever and tip arrangement and the application of a constant force at the sample surface.

The use of strain gauges has been attempted to provide a 24-hour IOP assessment using a contact lens-based approach. These devices attempt to position a strain gauge over the corneal limbus and correlate IOP fluctuation with changes in radius of curvature (R_{c}). However, the variability in human corneal shape makes it very difficult to properly position a strain gauge without customization and thus the correlation with true IOP has been poor.

There is a need in the art for devices, systems, and/or methods that can provide accurate monitoring and/or measurement of IOP over longer periods of time (e.g., up to 24 hours or longer) without requiring overnight stays in the hospital/clinical environment, preferably in a continuous or nearly-continuous manner, in order to better detect, treat, and manage glaucoma in patients.

### BRIEF SUMMARY

Discussed herein are various embodiments of a device for measuring or assessing one or more biomechanical properties of a cornea of a patient's eye, which may facilitate an assessment of intra-ocular pressure (IOP) of a patient's eye. Such a device may comprise a wearable contact lens, at least one cantilever sensor (e.g., a MEMS self-sensing cantilever or MEMS SSC) embedded in the contact lens, the cantilever sensor configured to measure elasticity of the cornea and/or an IOP of the eye, and at least one tip coupled to at least one cantilever sensor, the tip coupled to the MEMS SSC and configured to protrude beyond the posterior surface of the device to apply a force to the surface of the cornea. In some embodiments, a tip may be coupled to the cantilever that projects radially inwardly toward the center of the lens to behave as a backstop; such an arrangement may allow actively setting the cantilever to a “zero displacement” position, for example. A wearable contact lens device according to embodiments of this disclosure may provide a non-invasive device with the ability to monitor, assess, or measure the intraday fluctuations of IOP.

In some embodiments, the device includes a cantilever that is curved to conform to the contour of a contact lens. In some embodiments, the device may include a plurality of cantilever sensors disposed around the paracentral and mid-peripheral zones of the contact lens. Such a device may, for example, be configured to facilitate computing an average or composite elasticity of the cornea based on the elasticity measured by the plurality of cantilever sensors of the contact lens. An average or composite elasticity measurement may then serve as an input to a computation of IOP at a given point in time, for example.

In some embodiments, the contact lens of the device is a corneal contact lens. In some embodiment, the contact lens of the device is a scleral contact lens. In some embodiments, the contact lens of the device is a hybrid contact lens.

In some embodiments, the device further comprises an antenna coupled to at least one cantilever sensor (e.g., via an application specific integrated circuit or “ASIC”), the antenna configured to transmit data regarding measured elasticity and/or IOP to an external device. The antenna may, for example, be configured to transmit data to an external device wirelessly, for example via Bluetooth or NFC technology. The external device will then send data to a cloud database, and may also process the data before sending. In some embodiments, data transmitted via the antenna may include data from other components of the lens, including without limitation, data from an accelerometer, an analyte sensor, a thermometer (e.g., with thermal cut-off capability), etc. In some embodiments, data (e.g., elasticity data, accelerometer data, temperature data, etc.) may be stored in a data storage module of the device until it is transmitted via the antenna. The ASIC could, for example, control the coordinated functionality of each component of the lens and the flow of data between the data storage module and the antenna.

In some embodiments, the device further comprises a drug disposed within the contact lens, the drug configured to be eluted from the contact lens to the corneal surface (e.g., for absorption into the eye of the patient). For example, in some embodiments, the drug may be eluted based upon a measured elasticity value and/or IOP value exceeding a threshold.

In some embodiments, the device may be further configured to be calibrated to determine IOP based on one or more patient specific parameters (e.g., corneal topography, patient demographic information, etc.). For example, the device may be calibrated based on measured corneal thickness, or based on measured corneal radius of curvature, or based on a patient's age, gender, or other demographic information.

In some embodiments, a software application for using the contact lens-based device may include a patient interface configured to do one or more of the following in response to a measured elasticity value and/or IOP value exceeding a threshold: (a) contact a physician, (b) recommend initiation of, or a change in, therapy, or (c) make no changes.

In some embodiments, a software application for using the contact lens-based device may include a physician interface configured to do one or more of the following: (a) contact a patient, (b) change a therapy, or (c) make no changes.

While multiple embodiments are disclosed, still other embodiments will become apparent to those skilled in the art from the following detailed description, which shows and describes illustrative embodiments. As will be realized, the various implementations are capable of modifications in various obvious aspects, all without departing from the spirit and scope thereof. Accordingly, the drawings and detailed description are to be regarded as illustrative in nature and not restrictive.

### BRIEF DESCRIPTION OF THE DRAWINGS

FIG. 1 is a schematic cross-sectional view of a portion of an eye of a patient, illustrating the application of a known, external force to the cornea;

FIG. 2 is a plot showing the relationship between elasticity (Young's modulus) of the cornea and intraocular pressure (IOP) of the eye;

FIG. 3 is a conceptual system diagram showing the use of Atomic Force Microscopy (AFM) to measure elasticity, using a cantilever and tip arrangement and the application of a constant force at the sample surface;

FIG. 4 is an enlarged schematic view of portions of a system for using AFM to measure the elasticity of a sample (e.g., the cornea of an eye) using a cantilever arrangement;

FIG. 5A includes a side cross-sectional view of a contact lens device (with an enlarged inset image showing details thereof) with a biomechanical properties assessment device (e.g., MEMS SSC) disposed within the lens device, the lens device configured to be positioned over the cornea of a patient's eye, according to some embodiments;

FIG. 5B is a schematic top plan view of an exemplary contact lens that may be suitable for monitoring and/or assessing IOP in a patient according to some embodiments;

FIG. 5C is an enlarged side cross-sectional view of a cantilever tip arrangement for a cantilever sensor or self-sensing cantilever (or “SSC”) for use in the contact lens of FIG. 5B according to some embodiments;

FIG. 5D is an enlarged side cross-sectional view of an alternate embodiment of a cantilever tip arrangement for a cantilever sensor or self-sensing cantilever (or “SSC”) for use in the contact lens of FIG. 5B according to some embodiments;

FIG. 6 is a schematic perspective view of an embodiment of a contact lens with a biomechanical properties assessment device according to some embodiments;

FIG. 7 is a schematic representation of an embodiment of a contact lens with a biomechanical properties assessment device capable of communicating with a variety of exemplary external devices, according to some embodiments;

FIG. 8A is a high level flow diagram illustrating functionality of a graphical user interface (“GUI”) for a software application that may facilitate use of the contact lens device and/or systems by a Patient;

FIG. 8B is a high level flow diagram illustrating functionality of a graphical user interface (“GUI”) for a software application that may facilitate use of the contact lens device and/or systems by a Physician; and

FIG. 9 is a schematic perspective view of an embodiment of a contact lens with a biomechanical properties assessment device according to some embodiments;

FIG. 10 is schematic illustration of an embodiment of a contact lens with a biomechanical properties assessment device having an anterior layer (side view, left image), a measurement layer (front view, middle image), and a posterior layer (side view, right image), according to some embodiments;

FIG. 11A is a side view of an anterior lens layer of a lens device according to some embodiments;

FIG. 11B is a side view of a posterior lens layer of a lens device according to some embodiments;

FIG. 11C is a front view of a measurement layer of a lens device according to some embodiments;

FIG. 12 is an enlarged plan view of a cantilever module having multiple cantilever dies for use with a lens device according to some embodiments;

FIG. 13 is a graphical plot of regions or zones of a patient's eye;

FIG. 14A is a top plan schematic view of an exemplary contact lens according to some embodiments of this disclosure;

FIG. 14B is a partial, side cross-sectional view of the contact lens of FIG. 14A;

FIG. 14C is a top plan schematic view of an alternative contact lens according to some embodiments of this disclosure;

FIG. 15A includes a pair of corneal topography plots corresponding to a particular patient's eye for use with a device according to some embodiments of this disclosure;

FIG. 15B is an exemplary device for use with the corneal topography data in the plots of FIG. 15A, according to some embodiments of this disclosure;

FIG. 16A is a conceptual side cross-sectional view of a patient's eye with a force being applied at a surface of a cornea to illustrate various principles and techniques employed herein;

FIG. 16B is a conceptual schematic diagram of a custom AFM apparatus;

FIG. 16C is an image of a custom AFM apparatus;

FIG. 17A is a top perspective image of an artificial cornea to be used with a custom AFM apparatus;

FIG. 17B is a top perspective image of a pressure chamber of a custom AFM system;

FIGS. 18A-18G are a series of plots of experimental data showing displacement (indentation) versus pressure (IOP) corresponding to a range of varying spring constants;

FIG. 19 is a plot showing a relationship between indentation force as a function of indentation displacement or depth plotted for four different values of IOP for use with a device according to some embodiments of this disclosure;

FIG. 20A is a perspective image of a container for soaking an exemplary lens device in a medicated solution according to some embodiments of this disclosure;

FIG. 20B is a perspective image of an alternative container for soaking an exemplary lens device in a medicated solution according to some embodiments of this disclosure;

FIG. 20C is an enlarged cross-sectional image of a lens device having nanoparticles embedded with a drug for elution to a patient's eye, according to some embodiments of this disclosure;

FIG. 20D is schematic depiction of a lens device having a drug storage reservoir embedded therein for administration of a drug to a patient's eye, according to some embodiments of this disclosure;

FIG. 21A is a schematic diagram showing an embodiment of an external power source for a lens device according to embodiments of this disclosure;

FIG. 21B is a schematic diagram showing an embodiment of an internal power source for a lens device according to embodiments of this disclosure;

FIG. 22 is a block diagram illustrating a more detailed example of a computing device configured to perform the techniques described herein;

FIG. 23 is a flow diagram illustrating an example process for generating a patient interface, in accordance with one or more techniques of this disclosure; and

FIG. 24 is a flow diagram illustrating an example process for generating a physician interface, in accordance with one or more techniques of this disclosure.

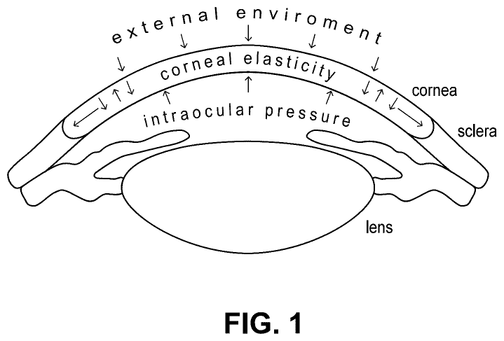

### DETAILED DESCRIPTION

The various embodiments herein relate to devices—including, for example, contact lenses—for assessing intra-ocular pressure of a patient's eye.

In certain embodiments, the device contemplated herein can be worn on a patient's eye like a standard contact lens. One source of complication and error in the assessment of IOP in known technologies relates to variations in central corneal thickness (CCT) among patients. Thin and thick corneas confound IOP assessment such that when IOP is corrected for CCT, 44% of normal tension glaucoma patients would be diagnosed as primary open angle glaucoma, and 30-65% of ocular hypertension patients would be diagnosed as normal. Another source of confusion is poorly defined physiologic IOP fluctuation. IOP changes continuously throughout the day such that there is approximately a 75% chance of not observing a patient's peak intraday IOP during normal clinical hours. A wearable IOP monitor according to any of the implementations herein would overcome these issues and therefore help facilitate a better understanding of physiologic and glaucomatous IOP patterns. For example, the various embodiments disclosed or contemplated herein overcome the inability of known technologies and measurement techniques to accurately measure and/or assess IOP over longer periods of time (e.g., up to 24 hours or longer).

One technique that may offer advantages in the measurement of IOP is Atomic Force Microscopy (“AFM”). AFM can be used to measure the elasticity of the cornea, which has been shown to correlate well with IOP.

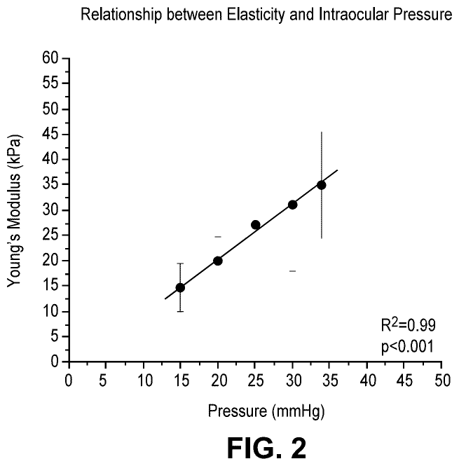

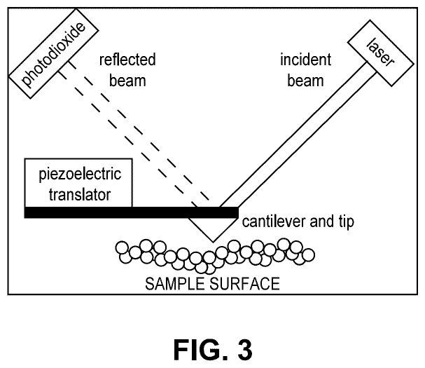

FIG. 2 is a plot showing the relationship between elasticity (Young's modulus) and intraocular pressure (IOP). As shown, there has been demonstrated a linear correlation between elasticity of the cornea and the IOP.

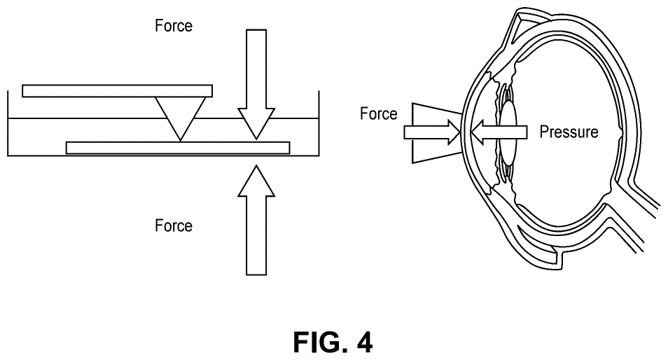

The various embodiments herein relate to wearable devices that utilize a MEMS SSC to measure biomechanical properties of a patient's cornea. For example, FIG. 4 is an enlarged schematic view of portions of a system according to one implementation for using AFM to measure the elasticity of a sample (e.g., the cornea) using a cantilever arrangement, where the cantilever deflection is measured to estimate and/or determine the elasticity of the sample. In some embodiments, one or more cantilever sensors or “self-sensing cantilevers” (SSCs) could be employed for this purpose by being embedded within a contact lens to form a wearable IOP monitor, for example. In some embodiments, a cantilever sensor may employ a MEMS SSC to measure or assess the elasticity of the cornea, and may in turn estimate and/or assess IOP using the correlation between elasticity of the cornea and IOP.

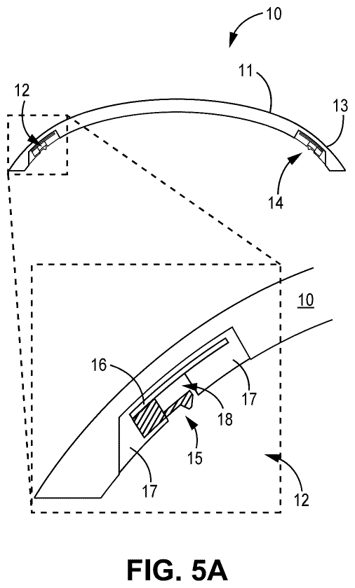

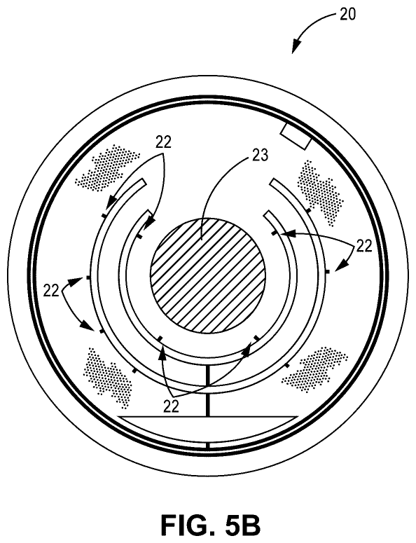

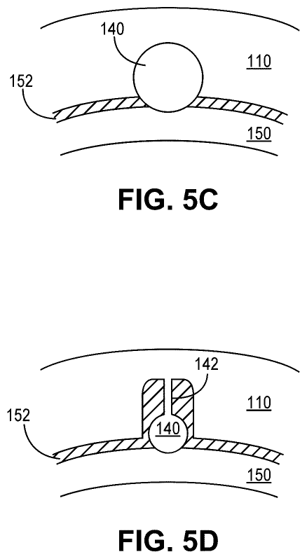

FIG. 5A is a side cross-sectional view showing an exemplary embodiment of a contact lens 10 containing a biomechanical properties assessment device 12 disposed therein, the lens 10 being configured to be positioned over the cornea of a patient's eye, according to some embodiments. The biomechanical properties assessment device 12 of the contact lens 10 may comprise an IOP assessment device 12, or the biomechanical properties assessment device 12 may be configured to provide an input to the calculation of IOP, in some embodiments. A portion of the lens 10 is enlarged in the lower inset image of FIG. 5A to show details of the biomechanical properties assessment device 12 according to various embodiments disclosed herein. For example, biomechanical properties assessment device 12 may comprise a cantilever assembly positioned in the lens 10 such that a cantilever tip of the cantilever assembly is configured to contact and/or apply a force to a surface of the cornea of the patient's eye. Also shown is a flexible electronics component and a hydrogel material disposed in a recess or housing or layer of the lens device 10. The hydrogel material may facilitate holding and positioning the flexible electronics and the cantilever assembly relative to the cornea. The flexible electronics may be configured to supply power to the cantilever assembly (e.g., for making measurements of one or more biomechanical properties of the cornea, for example), and/or for transmitting signals and data to and from the cantilever assembly.

With reference to the upper image of the lens device 10 of FIG. 5A, details of an exemplary “hybrid” contact lens 10 are shown according to certain embodiments. For example, in the embodiment of FIG. 5A, lens 10 may include a hybrid lens construction comprising an inner portion 11 coupled to an outer skirt portion 13, generally as depicted in FIG. 5A. In some embodiments, inner portion 11 may comprise a gas permeable lens, and outer skirt portion 13 may comprise a soft lens skirt. In some embodiments, outer skirt portion 13 may be formed to include one or more recesses in which a biomechanical properties assessment device 12 may be positioned. In some embodiments, an annular recess 14 may be formed in an inner/posterior surface of the outer skirt portion 13. Alternatively, a number of recesses 14 may be formed in an annular pattern around the outer skirt portion 13, for example. As shown in the exemplary embodiment of FIG. 5A, one or more biomechanical properties assessment devices 12 may be disposed at various positions along the recess 14. The biomechanical properties assessment device 12 may include a cantilever assembly 15, flexible electronics 16, and a hydrogel material 17 formed to hold the cantilever assembly 15 and flexible electronics 16 in place, while also creating an open or unenclosed area 18 that may allow for deflection of a cantilever tip portion of the cantilever assembly 15, for example.

Alternatively, various device embodiments herein can have more than one biomechanical properties assessment device (e.g., an IOP assessment device) disposed within a lens device of this disclosure. For example, FIG. 5B is a schematic top plan view of an exemplary contact lens 20 implementation with multiple biomechanical properties assessment devices 22 (IOP assessment devices 22) that may be suitable for monitoring and/or assessing biomechanical properties (and/or assessing IOP) in a patient. For example, the contact lens 20 as shown has a plurality of biomechanical properties assessment devices 22 integrated into the lens 20. In this example, the biomechanical properties assessment devices 22 may comprise cantilever sensors 22, and more specifically, they can be “self-sensing cantilevers” (SSCs) 22 integrated into the lens 20 and disposed in an array across the lens 20 generally as shown in FIG. 5B. (Note that, in the particular embodiment depicted in FIG. 5B, a greater number or a lesser number of SSCs 22 could have been alternatively used, as needed.) The use of multiple SSCs 22, arranged in an array for example, may be desirable to help address and/or account for variations in certain patient-specific geometries, such as variations in corneal thickness and/or radius of curvature across the corneal surface, either of which would likely affect the response of the cornea to IOP, and/or affect an estimation of IOP based on a measured elasticity of the cornea, for example. In the exemplary embodiment shown in FIG. 5B, the array of SSCs 22 can be disposed around an outer portion of the lens 20 area to minimize any potential interference with vision for the patient/wearer. For example, the array of SSCs 22 may be positioned to avoid the optical zone 23 to avoid visual interference. The array of SSCs 22 could be positioned in the paracentral zone or the peripheral transition zone, according to certain embodiments. In the embodiment of FIG. 5B, various electronics components (not depicted in FIG. 5B) may also be included and/or operably coupled to the array of SSCs 22. The electronics components could include, for example, any or all of the following elements without limitation: connectors, battery, power supply, capacitor, accelerometer, thermometer, data management, communications, processor, etc.

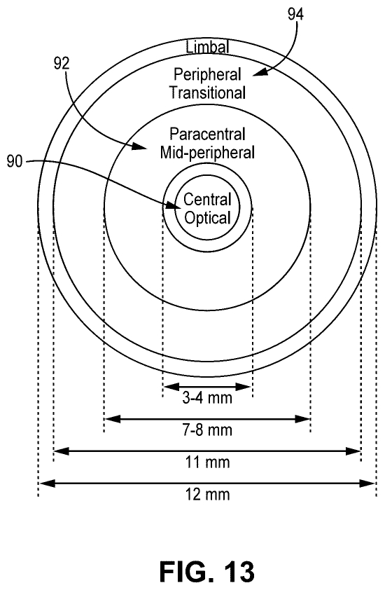

Central corneal thickness (CCT) is variable in humans and corneal thickness increases as you move toward the corneal periphery. Thus, in some embodiments, the SSC array may be positioned in the paracentral cornea, avoiding the central cornea so as not to interfere with the wearers' vision, avoiding the peripheral cornea where peripheral corneal vasculature and normal degenerations (e.g., arcus senilis, senile furrow) can potentially confound measurements. With reference to FIG. 13, the array of SSCs 22 may be disposed in the contact lens 20 such that, when worn by a patient, they are positioned outside of a central optical zone 90, for example in a paracentral mid-peripheral zone 92, or in a peripheral transition zone 94.

FIG. 5C is a schematic side cross-sectional view of a portion of a lens device 110 according to some embodiments, the lens device 110 having a tip portion 140 disposed within lens device 110 and configured to be placed in contact with a surface of a patient's cornea 150 in order to measure or assess a biomechanical property of the cornea 150. A tear film 152 is shown disposed over a surface of the cornea 150, and the tip portion 140 is shown extending through the tear portion 152 to apply a force directly to a surface of the cornea 150 to thereby assess a biomechanical property of the cornea 150.

Similar to FIG. 5C, FIG. 5D shows an alternate arrangement of lens device 110, according to some embodiments. In the embodiment of FIG. 5D, lens device 110 has a tip portion 140 extending from a cantilever portion 142. The cantilever portion 142 and the tip portion 140 are disposed within the lens device 110 and are configured to be placed such that the tip portion 140 is in contact with a surface of a patient's cornea 150 to thereby measure or assess a biomechanical property of the cornea 150. A tear film 152 is shown disposed over a surface of the cornea 150 and extending into a recess of the lens 110 housing the tip portion 140 and cantilever portion 142. The tip portion 140 is shown extending through the tear portion 152 to apply a force directly to a surface of the cornea 150 to thereby assess a biomechanical property of the cornea 150. In the embodiment of FIG. 5D (and in other embodiments depicted throughout this disclosure), the cantilever portion 142 (and possibly other portions) may be curved to follow the contour of the surface of the cornea 150.

Although it is contemplated that the SSC cantilevers and other on-board electronics components of the various implementations of the device may typically be placed in the paracentral zone of the lens, in certain alternative circumstances, transparent conducting electrodes/electronic connections (such as ITO) can be incorporated to make various connections on the sensors, and/or transparent materials such as silica/glass or polymers (or other comparable materials, possibly flexible and gas permeable) can be incorporated for certain portions of the cantilevers. Such transparent materials (and/or possibly translucent materials) can provide additional design flexibility in terms of placement of the SSCs in a suitable array on the contact lens, along with implementing additional antenna materials to improve the transmission capabilities of the device. In such embodiments, various elements of the device can be positioned in locations other than the paracentral zone of the lens.

In use, the device above can be operated in the following manner: actuation of the cantilever is used to induce a vertical force with the cantilever tip on the corneal surface of the eye. Stated differently, the actuator would bring the cantilever tip in contact with the corneal epithelium, indent to a known or predetermined depth or according to a known or predetermined force, and retract the tip to its original position. The negative deflection of the cantilever is what will be measured and used to derive elasticity measurements and/or IOP measurements. Actuation may be a mechanical actuation, or it may be a thermal actuation, or some other actuation mechanism, according to various embodiments. The negative deflection of the cantilever tip is an inverse measure for the stiffness of the eye model. Thus, the deflection of the cantilever tip can be sensed using piezoresistors in the SSC, for example. In certain exemplary implementations, thermal actuation current can be controlled using a computer or processor equipped with a data-acquisition interface. In some embodiments, two piezoresistors may be arranged in a Wheatstone bridge together with two thermal compensation resistors that are provided on the SSC device. The output voltage, Vₒ, can be amplified using an instrumentation amplifier and subsequently digitized using the same data acquisition interface.

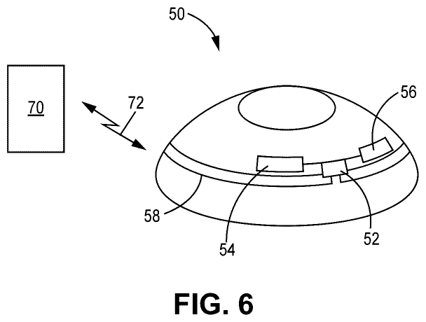

FIG. 6 is a schematic perspective view of a further embodiment of a contact lens 50 with a biomechanical properties assessment device according to some embodiments. FIG. 6 shows an embodiment of a contact lens-based device or system 50 showing a number of optional elements and/or features. For example, a battery 54 is shown operably coupled to an ASIC 52 to supply power thereto. Additionally, or optionally, a capacitor 56 is shown operably coupled to the ASIC circuit 52. In some embodiments, both a battery 54 and a capacitor 56 may be employed to provide power to the ASIC 52. For some operations, for example, a battery 54 may be able to store and deliver electrical energy to a capacitor 56, which may be configured to store and charge electrical energy in a manner that may increase the voltage available to perform an intermittent operation, such as a communication operation with an external device 70 (e.g., to transmit data 72 recorded by the contact lens 50 to the external device 70). For this purpose, an antenna 58 is also depicted in FIG. 6. In the particular embodiment shown, the antenna 58 is disposed as a pair of generally concentric rings near the outer periphery of the lens 50, operably coupled to the ASIC 52 as shown. The antenna 58 may be configured to transmit data 72 wirelessly to an external device 70 using known communication technologies, such as Bluetooth or other similar RF technologies, or infrared communications, or other wireless technologies.

With respect to the power supply (e.g., battery 54, capacitor 56, etc.), it may be appropriate to employ an energy conservation strategy in conjunction with making measurements, collecting data, transmitting data, etc. For example, continuous monitoring of IOP is desirable, but if enough data can be obtained by using an intermittent mode of data collection, for example, then this might be a way to extend the energy capabilities of the device 50, or alternatively, it may be a way to make the power supply portion of the device 50 smaller. An example of an energy conservation strategy may involve intermittent collection and/or transmission of data in “ON” and “OFF” periods. For example, a range of possible alternatives may include: 1 minute of data collection ON followed by transmission and 1 minute OFF; or 1 minute ON/transmission and 5 minutes OFF; or 1 min ON/transmission and 10 min OFF; or always ON, etc.

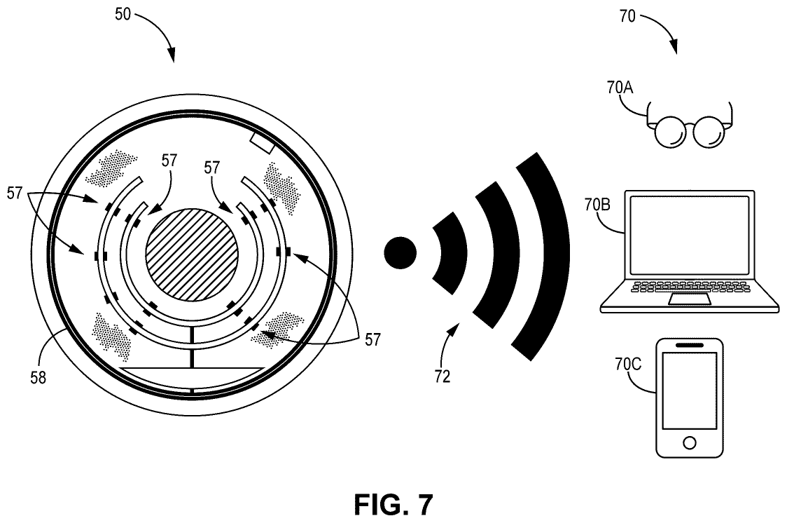

FIG. 7 is a schematic representation of an embodiment of the contact lens 50 of FIG. 6 having one or more biomechanical properties assessment devices disposed therein capable of communicating with a variety of exemplary external devices 70, according to some embodiments. For example, as depicted in FIG. 7, lens 50 may include a plurality of biomechanical properties assessment devices 57 and an antenna 58 configured to transmit data 72 wirelessly to an external device 70 using known communication technologies, such as Bluetooth, NFC, or other similar RF technologies, or other wireless technologies. FIG. 7 provides several examples of external devices 70 that may be configured to transmit and/or receive data 72 to/from lens 50. For example, an eyeglass frame 70A may be equipped with a communications module capable of transmitting and/or receiving data or other information wirelessly. Similarly, a laptop computer 70B or a cellular phone 70C are examples of external devices 70 that may be configured to communicate data, instructions, or other information to and from a contact lens device 50 of this disclosure.

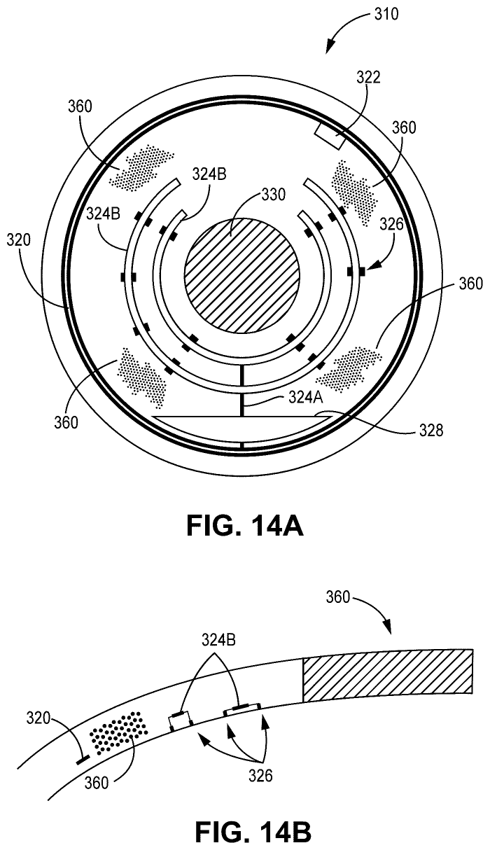

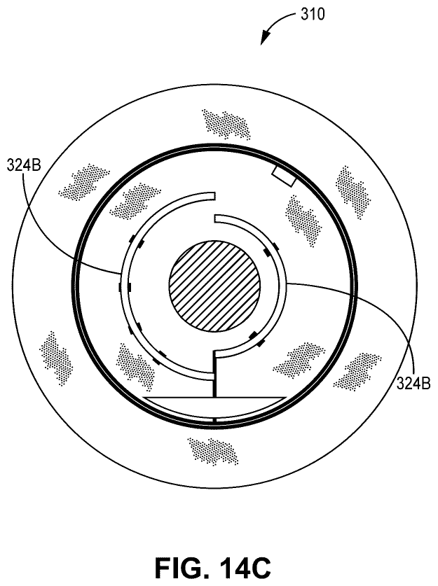

It should be noted that the lens embodiments of this disclosure may include an optical zone having certain refractive properties to aid a patient's vision. The optical zone (e.g., optical zone 23 of FIG. 5B, or optical zone 330 of FIGS. 14A and 14B) may be configured to include a prescription lens portion with refractive properties (e.g., varying refractive powers). An optical zone 23/330 may be added during manufacturing, for example, having refractive properties that are specific to a particular patient's vision prescription, according to some embodiments. The refractive power of the optical zone 23/330 may be selected to correct the patient's vision while they are wearing a lens device of this disclosure.

In some embodiments, it may be desirable to include a drug delivery aspect to the contact lens devices described above. For example, a drug delivery component (or drug compartment or compartments, etc.) may be housed within the material of the contact lens and may facilitate the ability of the lens to administer a drug (e.g., Timolol may be eluted for controlling IOP) to a patient's eye slowly and/or selectively. For example, the drug delivery component may incorporate a drug elution capability. In some embodiments, drug elution may be triggered based on an IOP measurement (e.g., a “theranostic” capability or approach). For example, certain threshold values for measured IOP may be established or set by a physician in advance, and upon exceeding the threshold, the device may be configured to elute the drug to the patient, or to increase the rate at which the drug is eluted, for example. A series of thresholds could be set, for example, to vary the delivery of the drug eluted from the lens. Alternatively, a more complex algorithm or profile could be set by the physician to control and/or vary the drug delivery regime. It is conceivable that a form of closed-loop feedback control could be established to increase and/or decrease drug elution in an attempt to control IOP in a manner well-suited for a particular patient, for example. Further details regarding a drug delivery functionality of the lens device of this disclosure is provided further below.

FIG. 7 is a summary of the background, theory, testing methods, and study results that support the development of a wearable sensor for assessing biomechanical properties of the eye, including determining elasticity of the cornea and/or assessing IOP. It should be noted that this application and embodiments herein are not intended to be limited by any particular theory that may be set forth herein.

Preliminary research and results indicate that indentation measurements performed at the corneal surface using an AFM cantilever and an SSC are directly correlated to intraocular pressure. It is believed that a complex interrelationship between CCT (a given patient's corneal thickness), R_{c} (radius of curvature), YM (Young's Modulus of elasticity), and IOP (intraocular pressure) can be determined empirically (e.g., through experiments and testing, etc.) and may be used to develop an analytical model that relates these parameters.

Before fitting a patient with the wearable sensor, measurements may be acquired to set the baseline on the sensor, thereby calibrating the output. For example, a patient's corneal thickness (CCT) and radius of curvature (R_{c}) may be measured. Then, a biomechanical property of the eye, such elasticity of the cornea (e.g., Young's Modulus or YM) may be assessed using a wearable contact lens-based SSC system, as described herein, and the resulting data would be processed by the analytical model to produce an accurate assessment of the patient's IOP, for example, thereby providing better clinical outcomes with respect to glaucoma detection and/or treatment. In some embodiments, the initial measurements or assessments of patient-specific parameters, such as age, gender, CCT and R_{c}, may be used to calibrate the output of the wearable device such that it is configured to provide an accurate measurement of IOP as a function of time.

Several mathematical models that relate to determining IOP may be employed:
(a) Hertz model for a cantilever with a spherical indenter tip:

$$F = \frac{4 E \sqrt{{R}_{AFM}}}{3 ( 1 - {v}^{2} )} {D}^{3 / 2}$$

and
(b) Modified Orssengo and Pye model for IOP calculation:

$$E = \frac{1}{7500} ( \frac{3 F ( 1 - {v}^{2} )}{4 \sqrt{{R}_{AFM}}} {D}^{- 3 / 2} - \frac{{{R}_{C} ( {R}_{C} - \frac{1}{2} CCT )}^{2} ( 1 - v )}{{R}_{AFM}^{2} CCT} IOPT )$$

where E is the modulus of elasticity of the cornea, F is the force measured by the cantilever, v is the Poisson ratio of the cornea (v=0.49), RAFM is the radius of the cantilever tip (e.g., 25 μm in these experiments), D is the measured depth of indentation, R_{c} is corneal radius of curvature, CCT is corneal thickness, and IOPT is the “true” IOP. CCT and R_{c} may come from Optical Coherence Tomography (“OCT”) measurements taken of each cornea, for example. E may be quantified from measurements on human corneas while not under pressure. For each value of pressure, the values of F and D measured from the AFM cantilever, SSC, or custom sensor will be plugged into the formula to calculate IOPT. For traditional AFM cantilevers, F and D will come directly from the measurements; for SSCs, F will be determined by converting the measured voltage and D will come from the settings of the device; for the new custom sensors, F will be calculated from the measured bridge voltage and D will come from the actuation current. The calculated value of IOPT will be compared to the reading from the pressure sensor. The coefficients in the model will be adjusted accordingly so that the IOPT outputted from the analytical model matches the reading from the pressure sensor for all values of pressure. In the end, three different analytical models (traditional AFM, SSCs, new custom sensor) will be developed to relate patient specific parameters (age, gender, CCT and R_{c}) to IOPT based on the outputs of the sensors.

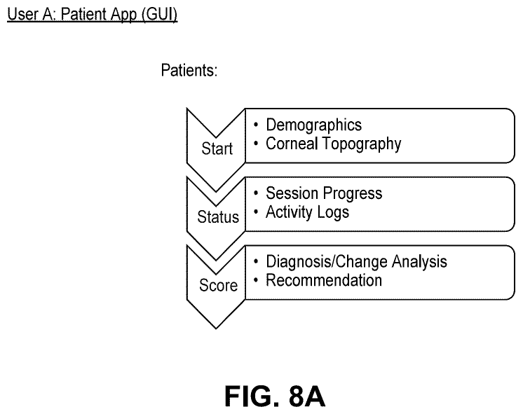

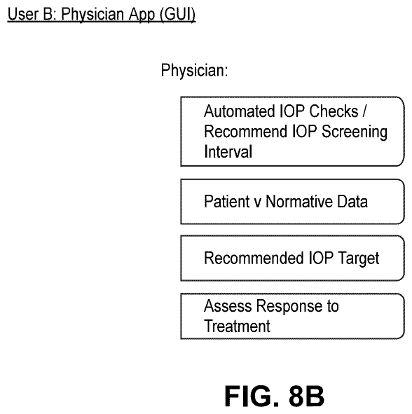

FIG. 8A is a high level flow diagram illustrating functionality of a graphical user interface (“GUI”) for a software application that may facilitate use of the contact lens device and/or systems by a Patient or individual user. The Patient App and/or GUI might facilitate entering data that is specific to a particular patient/user (e.g., patient demographic data, clinical measured data such as corneal topography, etc). In some cases, the Patient App and/or GUI may be configured to “auto-populate” certain patient information upon logging in or registering, for example; in such cases, patient data previously uploaded by a physician could link to the Patient App upon verifying the patient's identity, etc. The Patient App and/or GUI may also enable a user to activate certain features such as data collection, calibration, reporting, etc. The Patient App and/or GUI may be configured to provide alerts (e.g., potential diagnoses), recommendations (e.g., changes to treatment), or other guidance. In some embodiments, a Patient App and/or GUI may include indicators, alerts, or prompts to do any or all of the following actions, as needed: 1) Contact the prescribing physician for further evaluation and possible change in therapy; 2) Recommend the next appropriate screening/monitoring interval; or 3) Urgently contact a Physician. In some embodiments, a Patient App and/or GUI may be configured to provide a simplified indication (e.g., a color-coded display) that quickly signifies the nature of the prompt to the Patient. A Green, Yellow, or Red color scheme may be used in some embodiments to quickly convey the importance or urgency of the information to the patient, for example.

FIG. 8B is a high-level flow diagram illustrating functionality of a graphical user interface (“GUI”) for a software application that may facilitate use of the contact lens device and/or systems by a Physician or clinician. The Physician App and/or GUI might, for example, include features that enable a physician to monitor several different patients, or to selectively monitor a specific patient, for example. The Physician App and/or GUI may enable a physician to analyze data collected by a patient, or activate certain features, including conducting an IOP check (e.g., manually or automated), viewing patient data vs. normative data, recommending an IOP target level, recommending a treatment or a change in the current treatment, evaluating the response to a treatment protocol, etc. A Physician App and/or GUI interface may, for example, include a “global view” that enables a Physician to see the status of all such patients, and may include the ability to filter or sort the data in order to help detect or identify: 1) patients that need urgent attention (e.g., to prompt them to schedule a clinical evaluation, or push information to such patients urgently); 2) patients with less urgent conditions, for example, those that may require a medication change (e.g., this may also push instructions/prescriptions to such patients); and 3) patients that are ok and who should continue on their current treatment regimen without modification.

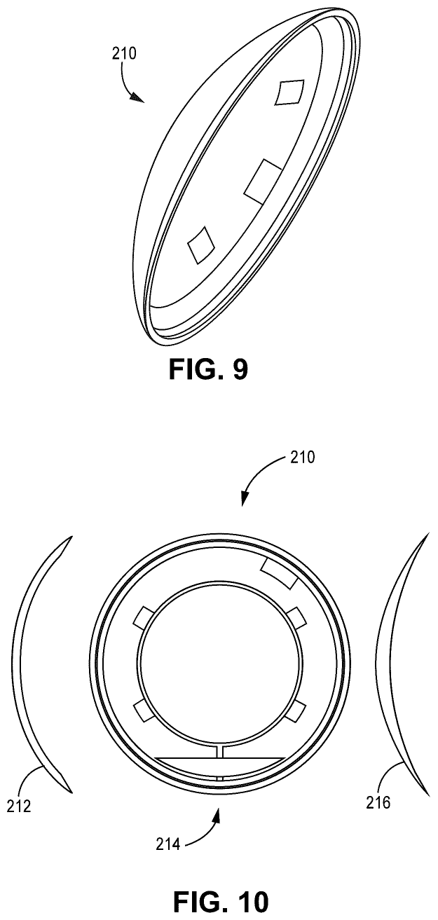

As shown in a representational image in FIG. 9, various embodiments of this disclosure relate to a soft contact lens 210 for measuring a biomechanical property (e.g., corneal elasticity) of a wearer's eye via an embedded array of measurement devices (also referred to herein as “cantilevers,” “cantilever sensors,” and “self-sensing cantilevers” or “SSCs”), wherein the lens 210 is intended for multiple uses.

As shown in FIG. 10, the lens 210 has three layers: (1) the anterior lens layer 212, (2) the measurement layer 214 (shown in front view), and (3) the posterior lens layer 216. The measurement layer 214 is disposed between the two lens layers 212 and 216, with the two lens layers 212, 216 bonded together. In a particular exemplary embodiment, the total thickness of lens 210 is <600 μm in the center of the lens 210, with contoured edges around the outer perimeter of the lens 210 and a total lens 210 diameter of 14 mm.

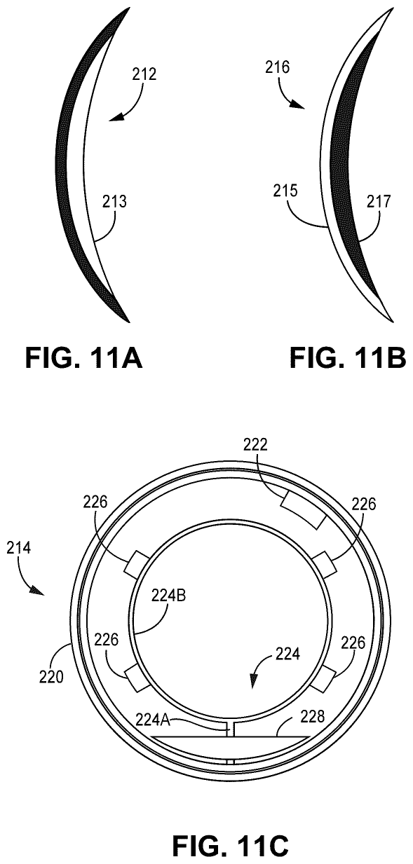

As shown in FIG. 11A, the anterior lens layer 212 is made of silicone hydrogel, which provides oxygen transmissibility and heat dissipation. The posterior surface 213 of the anterior lens layer 212 is contoured to accommodate mounting of the measurement layer 214 and bonding with the posterior lens layer 216.

Similarly, as shown in FIG. 11B, the posterior lens layer 216 is made of silicone hydrogel as well. Further, the anterior surface 215 of the posterior lens layer 216 is also contoured to accommodate mounting of the measurement layer 214 (such that the measurement layer 214 is disposed between the two outer layers 212, 216) and bonding with the anterior lens layer 212. With reference to FIG. 11B, the posterior surface 217 of the posterior lens layer 216 may have openings that permit the extension of the cantilever tips beyond the posterior surface 217 of the posterior lens layer 216 toward the cornea.

The silicone hydrogel and construction of the anterior and posterior lens layers 212, 216 may provide sufficient stiffness to safely house the measurement layer 214, while also providing sufficient flexibility for manipulation by the wearer. More specifically, the silicone hydrogel may be formulated (e.g., modified) to achieve a desired level of stiffness and/or flexibility. It should be noted that, in some cases, the stiffness of the lens layers 212, 216 may be an additional factor to account for when measuring elasticity, and/or when using a measured elasticity value to calculate IOP.

With reference to FIG. 11C, the measurement layer 214 has an antenna ring 220, a wireless communication module 222 attached to the antenna ring 220, and an array support structure 224 attached to the antenna ring 220. The array support structure 224 has an arm 224A attached to the antenna ring 220 and a semicircular support ring 224B disposed within the antenna ring 220 and attached to the arm 224A (and thereby attached to the antenna ring 220). According to one embodiment, the support structure 224 can be made of gold or any other metal with similar characteristics. In some embodiments, the support structure 224 may comprise flexible electronic circuits that may be configured to provide power to the individual SSCs and/or other modules, and may be further configured to transfer data from an SSC to a data storage module and antenna as controlled by an ASIC 228.

With continued reference to FIG. 11C, attached to the inner support ring 224B are four self-sensing cantilever modules 226 (or cantilever sensors 226), each of which is configured to measure elasticity of a portion of the patient's eye (e.g., the cornea). Alternatively, 2, 3, 5, 6, 7, 8, 9, 10, or any number of cantilevers 226 that can fit on the support ring 224B can be provided. The support ring 224B and the cantilevers 226 are disposed within the lens 210 and thus adjacent to the patient's eye (when the lens 210 has been placed on the patient's eye) in the mid-peripheral (paracentral) zone 92 and/or the peripheral (transitional) corneal zone 94 such that they do not block the vision of the patient. Each cantilever sensor or module 226 can have a predetermined or tunable spring constant. The cantilever tip associated with each cantilever module 226 may be spherical (or rounded, or of other suitable geometric shape) with a diameter ranging between 10-50 microns, and the total travel distance from rest to corneal indentation may vary from about 5-10 microns, according to some embodiments. The cantilever sensors 226 may include a wheatstone bridge in some embodiments; in such embodiments, the cantilever sensors 226 may be considered “self-sensing” because they have the ability to sense deflection of the cantilever or cantilever tip without the use of a laser.

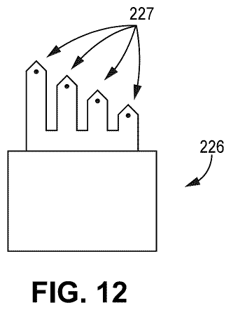

FIG. 12 shows an enlarged view of a cantilever module 226 having several cantilever dies 227 associated with a given cantilever module 226, according to some embodiments. In such embodiments, the multiple cantilever dies 227 may have differing characteristics, such as varying lengths (as generally shown in FIG. 12) and/or varying spring constants, for example, to provide greater measurement flexibility or accuracy.

Referring again to FIG. 11C, the device 210 (e.g., measurement layer 214 of device 210) may also have an application-specific integrated circuit (ASIC) 228 that is coupled to each of the cantilever modules 226 and the wireless module and antenna 222 via flexible electronics 224. More specifically, the flexible electronics may extend from the ASIC 228 to the cantilever modules 226 via the arm 224A and the support ring 224B, and may further extend from the ASIC 228 to the wireless module and antenna 222 via the antenna ring 220. As such, the ASIC 228 can power the lens 210 (e.g., provide power to the cantilevers 226 and any other onboard components), manage the data acquisition protocol of each self-sensing cantilever 226, store the data collected therefrom, and transmit the data to the wireless communication module 222. In some embodiments, the ASIC 228 has a battery with a power capacity of 72 hours and a data capacity of 14 days' worth of data.

In one embodiment, the wireless communication module and antenna 222 can receive data from the ASIC 228 with either episodic or continuous data transfer. In some embodiments, episodic data transfer may be facilitated via use of an external power supply, but it may also be facilitated via use of an internal power supply in other embodiments. Similarly, continuous data transfer may be facilitated via use of an internal power supply, but it may also be facilitated via use of an external power supply in other embodiments. The choice of internal or external power supply may be driven by factors including, for example, considerations of heat generated by transmission of data, size and availability of suitable power supplies, etc. The communication format can be either via Bluetooth or NFC with requisite encryption for transmitting protected health information (“PHI”). In certain embodiments data can be transferred to a proprietary web-based User Interface or, in other embodiments, to an app-based user interface.

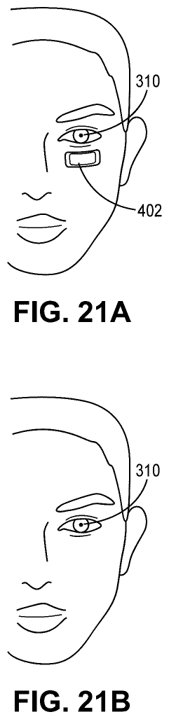

As noted above, the power supply could be external, using inductive power with intermittent data transfer (for example, cycling through ON→Measure→Transmit→OFF). In one embodiment, the external supply could be positioned under the eye like the patches often used by athletes. Alternatively, the power supply could be internal, with internal data storage. FIG. 21A shows an example of an external power supply 402 worn as a patch beneath a patient's eye in conjunction with a lens device 310 according to embodiments of this disclosure. Note that although the specific patch configuration shown in FIG. 21A is positioned below the eye of the patient, other configurations could be employed, including a circular patch configuration (around the eye socket area), or an eyelid patch, or some other form of sticker placed on the patient. Other possible examples of external power supplies 402 may include devices worn by the patient, include eyeglass configurations and the like. In some embodiments, external power supply 402 may be configured to periodically pulse (or pulse when needed) to provide electrical power to operate the lens device 310. FIG. 21B shows an example of a lens device 310 having an internal power supply (not shown) according to embodiments of this disclosure. Such embodiments may have a battery and/or a capacitor arrangement, for example, within the structure of the lens device 310 itself, for example.

The contact lens device 310 may be configured, in some embodiments, to incorporate certain safety features. For example, a temperature sensor may be provided as part of lens 310 so that the lens device 310 may be configured to shut off or reduce signal transmissions in order to avoid generating heat. A predefined temperature limit may be set (and/or may be adjustable) for this purpose, according to some embodiments.

FIG. 14A is a top plan schematic view of an exemplary contact lens 310 according to some embodiments of this disclosure. Similar to the embodiments depicted and described above with respect to FIGS. 9 through 11C, the contact lens 310 of FIG. 14A may include anterior and posterior lens layers formed of silicone hydrogel, with a measurement layer sandwiched therebetween. For example, contact lens 310 includes an antenna ring 320, a wireless communication module 322 attached to the antenna ring 320, and an array support structure attached to the antenna ring 220. The array support structure may include an arm 324A attached to the antenna ring 320, and a semicircular support ring 324B disposed within the antenna ring 320 and attached to the arm 324A (and thereby attached to the antenna ring 320). In some embodiments, more than one support ring 324B may be employed, as indicated by the two support rings 324B shown in the exemplary embodiment of FIG. 14A. This may enable positioning of the cantilever modules 326 at varying distances (e.g., varying radial distances, for example) from the central optical zone 330 to refine the measurement results, as will be described more fully below. According to one embodiment, the support structure 324A and/or 324B may be made of gold or any other metal with similar characteristics.

Attached to the support ring(s) 324B are a plurality of self-sensing cantilever modules 326 (or cantilever sensors 326), each of which is configured to measure elasticity of a portion of the patient's eye (e.g., the cornea). As noted above, any number of cantilevers 326 can be arranged on the support rings 324B, according to various embodiments. The support rings 324B and the cantilever sensors 326 are disposed within the lens 310 and thus adjacent to the patient's eye when worn (e.g., when the lens 310 has been placed on the patient's eye) in the mid-peripheral (paracentral) zone and/or the peripheral (transitional) corneal zone such that they do not block the vision of the patient; in some embodiments, the support rings 324B and the cantilever sensors 326 are disposed such that they are radially outside of the central optical zone 330. Each cantilever sensor or module 226 may have a predetermined or a tunable spring constant. The cantilever tip associated with a given cantilever module 326 may be spherical (or rounded, or of other suitable geometric shape) with a diameter ranging generally between about 10-50 microns, and the total travel distance from rest to corneal indentation may vary from about 5-10 microns, according to some embodiments. The cantilever sensors 326 may include a wheatstone bridge according to some embodiments; in such embodiments, the cantilever sensors 326 may be “self-sensing” because they may have the ability to sense deflection of the cantilever or cantilever tip without the use of a laser.

The lens device 310 of FIG. 14A may also include an application-specific integrated circuit (ASIC) 328 that is coupled to the cantilever modules 326 and to the wireless module and antenna 322 via flexible electronics, for example. The flexible electronics may, for example, extend from the ASIC 328 to the cantilever modules 326 via the arm 324A and via the support ring(s) 324B, and may further extend from the ASIC 328 to the wireless module and antenna 322 via the antenna ring 320. As such, the ASIC 328 can power the lens 310 (e.g., provide power to the cantilevers 326 and to other onboard components), manage the data acquisition protocol of each self-sensing cantilever 326, store the data collected therefrom, and transmit the data via the wireless module and antenna 322. In some embodiments, the ASIC 328 has a battery with a power capacity of 72 hours and a data capacity of 14 days' worth of data.

In one embodiment, the wireless communication module and antenna 322 can receive data from the ASIC 328 with either episodic (e.g., using an internal power supply) or continuous data transfer (e.g., using an external power supply). The communication format can be either via Bluetooth or NFC with requisite encryption for transmitting protected health information (“PHI”) in some embodiments. In certain embodiments, data can be transferred to a proprietary web-based User Interface or, in other embodiments, to an app-based user interface.

The lens device 310 of FIG. 14A may also include one or more drug delivery components 360 disposed on or within the contact lens 310. As shown, a plurality of drug delivery components 360 may be disposed in portions of the contact lens 310 such that a drug (e.g., Timolol may be used in some embodiments for controlling IOP, for example) is configured to be eluted from the contact lens 310 to the patient's eye. In some embodiments, the drug may be eluted from drug delivery components 360 based upon a measured elasticity value and/or an assessed/computed IOP value exceeding a threshold.

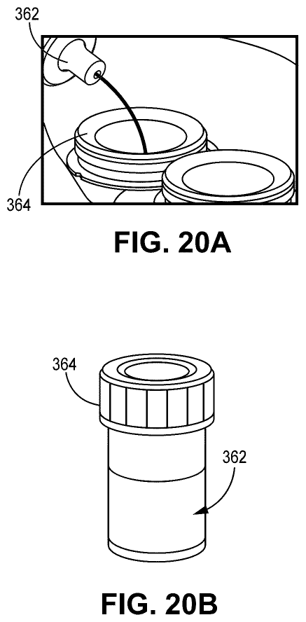

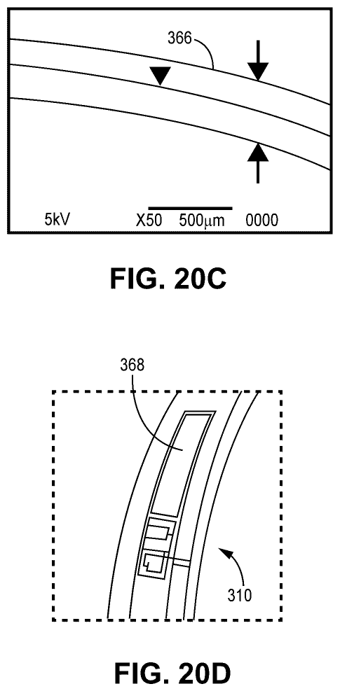

Several different techniques may be used to provide the drug delivery components 360 for the lens device 310. A first technique may involve soaking the lens 310 with a medicated solution such that the medicated solution is later eluted from the drug delivery component 360 to the patient's eye while being worn. The soak process may be performed during storage of the lens device 310 (e.g., during manufacturing, prior to shipping commercially, etc.) according to some embodiments. Lenses may be shipped to the patient in an appropriate medicated solution to ensure sterility, efficacy, etc., or in some cases, a manufacturer may label and send the solution to patients. Based on measurements made by the sensors 326 of the lens device 310, a physician or other healthcare practitioner may begin a periodic soak of the lens device 310 in an appropriate medicated solution, or the physician may prescribe that a lens device 310 be delivered to the patient pre-soaked in the appropriate medicated solution, for example. Alternately, a software application may alert the patient to begin a soak protocol in response to detecting a measured value from the lens device 310 that is beyond some predetermined threshold amount. FIGS. 20A and 20B show images of two embodiments of a container 364 that could be used for performing a soak of the lens device 310 in a medicated solution 362. Soaking the lens 310 in the medicated solution 362 may thereby provide the lens 310 with a drug delivery component 360 capable of eluting the drug to the patient's eye when later worn by the patient. It should be noted that the container 364 may be the same container used for normal daily cleaning/sanitation of the lens device 310, according to some embodiments. It should also be noted that in some embodiments, container 364 may be configured to perform additional functions relative to the operation of the lens device 310. For example, in some embodiments, container 364 may enhance the ability to clean the lens device 310, for example by adding an ultrasonic mode for helping to remove debris from the device 310 (including from the cantilevers), or by providing a ultraviolet (UV) exposure feature in the container 364 for enhanced cleaning capability. In other embodiments, the container 364 may be configured to provide electrical power to the lens device 310. For example, the container may be configured to charge a battery of the lens 310, or deliver a charge for storage on a capacitor of the lens 310. In some cases, such charging could take simultaneously with soaking of the device 310 (either in cleaning solution or in medicated solution, for example). In some further embodiments, container 364 may include a communications capability. For example, container 364 could be configured to download stored data (e.g., biomechanical measurement results obtained by a cantilever sensor, for example) from the device 310, or transmit certain information (e.g., patient specific parameters, corneal topography details, physician-prescribed parameters, etc.) to the device 310 or to an external device. The container 364 may, for example, communicate with lens device 310 using a wired or wireless technology (e.g., Bluetooth, NFC, etc.). Such communication may be triggered automatically in some embodiments, for example upon placement of the lens device 310 within container 364, or upon actuation of a button on the container 364, as possible examples.

A second technique for providing the drug delivery components 360 may involve the placement of “nano-particles” in the lens 310 during the manufacturing process, for example. FIG. 20C is a greatly enlarged cross-sectional image showing placement of exemplary nano-particles 366 within one or more of the layers of the lens device 310, or between such layers. Nano-particles 366 may be configured to break down over time to deliver an appropriate drug from the drug delivery component 360 to the patient's eye. It may be possible, in some embodiments, to customize the porosity of a silicone hydrogel material of lens 310 such that it facilitates loading of the drug nano-particles 366 into the lens 310, and/or facilitates controlling the rate of delivery of the drug to the patient's eye, for example.

A third technique for providing the drug delivery components 360 may involve the use of one or more drug storage reservoirs 368 positioned within the lens 310, for example. A schematic representation of such a drug reservoir 368 is provided in FIG. 20D. In such embodiments, a drug storage reservoir 368 may comprise a container or “sack” of medicated solution (e.g., Timolol may be provided in the storage reservoir 368 in some embodiments for controlling IOP, for example). A drug reservoir 368 may also be configured to trigger the release of a drug when needed, for example in response to a measured or assessed biomechanical property of the eye exceeding some threshold or meeting some criteria. In some embodiments, a customizable therapy may be implemented such that multiple drugs (e.g., a customized “cocktail” of medications) may be released from a plurality of drug reservoirs 368; the types of drugs and the amounts may be configured to vary depending on predefined or customized criteria (including things like measured/assessed biomechanical properties of the eye exceeding some threshold criteria, history of recent therapies, time of day, etc.).

FIG. 14B is a partial, side cross-sectional view of the lens 310 of FIG. 14A. FIG. 14B shows how certain components of the lens 310 may be arranged relative to a cross-sectional thickness or width of lens 310. For example, as shown in FIG. 14B, the cantilever sensors 326 (or sensor modules 326) may be disposed proximate the corneal facing surface of lens 310 such that a cantilever tip of a cantilever sensor is positioned in contact with an epithelial surface of the patient's cornea when the lens 310 is worn by a patient.

FIG. 14C is a depiction of an alternate embodiment of the lens 310. For example, in FIG. 14C, the lens 310 has two semicircular support rings 324B; however, they are not as concentric or parallel to each other as are the support rings 324B of FIG. 14A. The particular configuration of support rings 324B shown in FIG. 14C may be chosen as being suitable for a particular patient (e.g., based on specific corneal topography, for example). However, the configuration of FIG. 14C is exemplary only, and many similar alternative arrangements of support ring (or rings) 324B may be chosen for a particular situation by one or ordinary skill in this field.

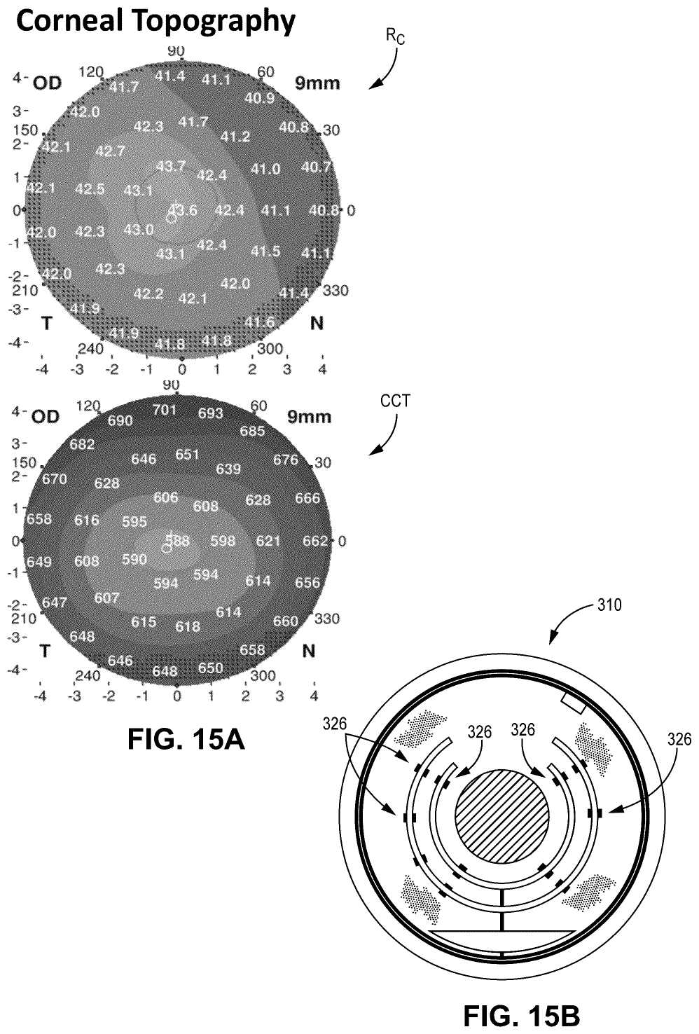

FIG. 15A shows a pair of corneal topography plots corresponding to a particular patient's eye. An exemplary contact lens 310 depicted in FIG. 15B may be configured or customized for use by the particular patient by accounting for the information in the corneal topography plots of FIG. 15A that are specific to the given patient. For example, the upper plot of FIG. 15A is a mapping of the radius of curvature (R_{c}) across a corneal surface of the patient's eye. Likewise, the lower plot of FIG. 15A is a mapping of the corneal thickness (CCT) for this patient. Other patient-specific parameters may also be measured and plotted for use in a similar manner to that described below.

The mapping of the plots in FIG. 15A may be taken at an initial patient visit or at a follow-up evaluation, for example, where such measurements may be obtained for use by the contact lens 310 and/or associated components and software to more accurately compute IOP from the biomechanical properties of the patient's eye measured by the lens 310. In some cases, the corneal topography mappings of FIG. 15A may enable a measurement made by a cantilever module 326 (e.g., an elasticity measurement) to be used to compute another parameter, such as intra-ocular pressure (IOP), while taking into account the specific aspects of corneal topography, such as R_{c} and CCT, at the location corresponding to the specific cantilever module 326.

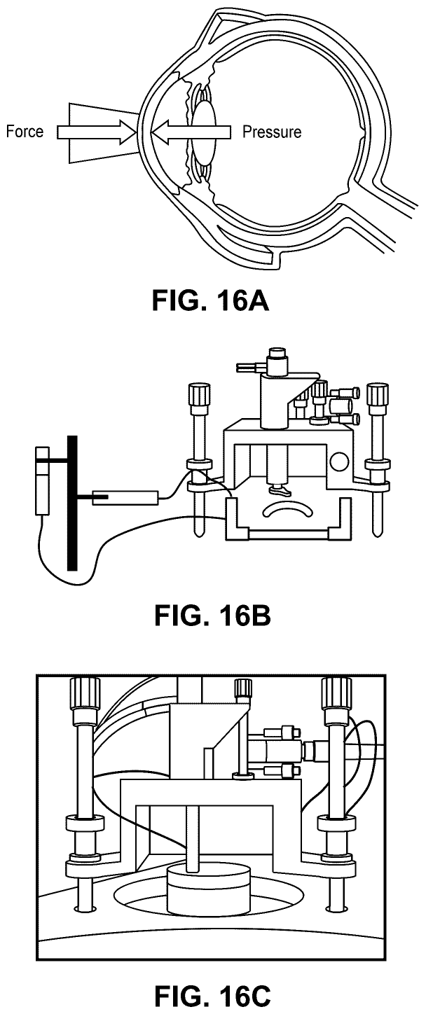

Atomic Force Microscopy, or AFM, is a high-resolution, high-sensitivity technique used to measure mechanical properties of biological samples. Traditionally, AFM measurements of mechanical properties of samples are performed on rigid substrates. But previous studies have shown that a cantilever (e.g., a cantilever sensor) may be sensitive to a rigid substrate in the case of thin samples, and the measurements of Young's modulus of elasticity indicate a greater degree of stiffness than they should because of this perceived force and/or rigidity from the substrate interaction. We wanted to take advantage of this phenomenon. We hypothesized that if we applied the same force on the surface of the cornea of the eye, the perceived measurement of Young's modulus would change as the force of pressure from within the eye changed. This concept is indicated as shown in FIG. 16A.

A custom AFM apparatus designed by Dr. Noel Ziebarth at the University of Miami was used in proof-of-concept experiments in porcine eyes to validate this hypothesis. A conceptual schematic diagram of the custom AFM apparatus is depicted in FIG. 16B; an image of the AFM system used in the experiments is provided in FIG. 16C. The results showed that there is a relationship between AFM measurements at the surface of the cornea and intraocular pressure on the opposite side. These measurements formed the basis of a previous patent application, PCT/US21/17919, the contents of which are incorporated by reference herein in relevant part.

The AFM cantilever is the active component of the AFM system in mechanical studies. The cantilever comes into contact with the sample and undergoes a combination of indentation and deflection, depending on the stiffness of the sample. Selection of the cantilever tip diameter and spring constant are aspects of the experimental design. Previous experiments in the lab had shown that a spring constant of 1.75 N/m was sufficient to measure human corneas mounted on a rigid substrate; however, measurements of epoxy and aluminum required a spring constant of at least 40 N/m. Based on previous experience, we hypothesized that the spring constant was a significant design constraint in the development of an AFM cantilever-based IOP measurement system. We designed an experiment to determine the role of cantilever spring constant.

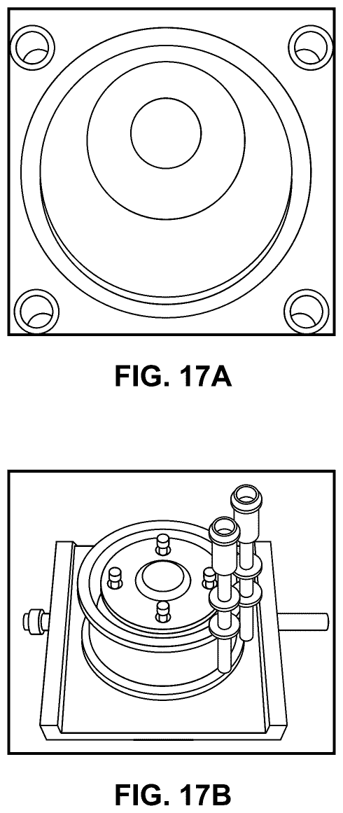

In these experiments, an artificial cornea (Cordelia, Bioniko), as shown in the image of FIG. 17A was used to minimize variability inherent to tissue experimentation. Prior to use, the artificial cornea(s) were rehydrated with warm (75-95° F.) deionized water for 10 minutes. The artificial cornea was then mounted in a custom developed pressure chamber, as shown in the image of FIG. 17B, and traditional AFM measurements were performed at pressures ranging from 15-39 mmHg, in increments of 2 mmHg. Fifteen repeat measurements were performed at each pressure increment. Using custom MATLAB code, the difference in displacement from contact until 1V of force was calculated. For each pressure increment, statistical outlier analysis was performed on the displacement values, and outliers were omitted.

FIG. 17A: Artificial cornea (Cordelia) used for additional experiments.

FIG. 17B: Custom pressure chamber designed for these experiments. The artificial cornea was mounted within the chamber, and pressure was increased incrementally, while measuring output from the SSC.

The cantilevers used were standard tipless cantilevers sold by MikroMasch: HQ: NSC36 and HQ: NSC35. Each of these cantilever chips contain three cantilevers with different spring constants. The nominal spring constants of the HQ: NSC36 chip are 0.6/1/2 N/m and the nominal spring constants of the HQ: NSC35 chip are 5.4/8.9/16 N/m. The “true” spring constant of each cantilever was measured using the Thermal Power Spectral Density (PSD) technique on an Asylum MFP-3D AFM. Thirteen different cantilevers were used in these experiments (Table 1). Of note, the nominal spring constants of cantilevers from the HQ: NSC36 chips were more closely aligned with the measured spring constant than those from the HQ: NSF35 chip. A Bland-Altman analysis showed that the deviation from nominal spring constant is significantly related to nominal spring constant.

TABLE 1

| Nominal Spring | Measured Spring |
|---|---|
| Constant (N/m) | Constant (N/m) |
| 0.6 | 0.6 |
| 0.6 | 0.6953 |
| 1 | 1.143 |
| 2 | 2.1 |
| 2 | 2.178 |
| 2 | 2.181 |
| 2 | 2.194 |
| 2 | 2.586 |
| 8.9 | 6.139 |
| 8.9 | 6.44 |
| 16 | 1.939 |
| 16 | 9.702 |
| 16 | 10.935 |

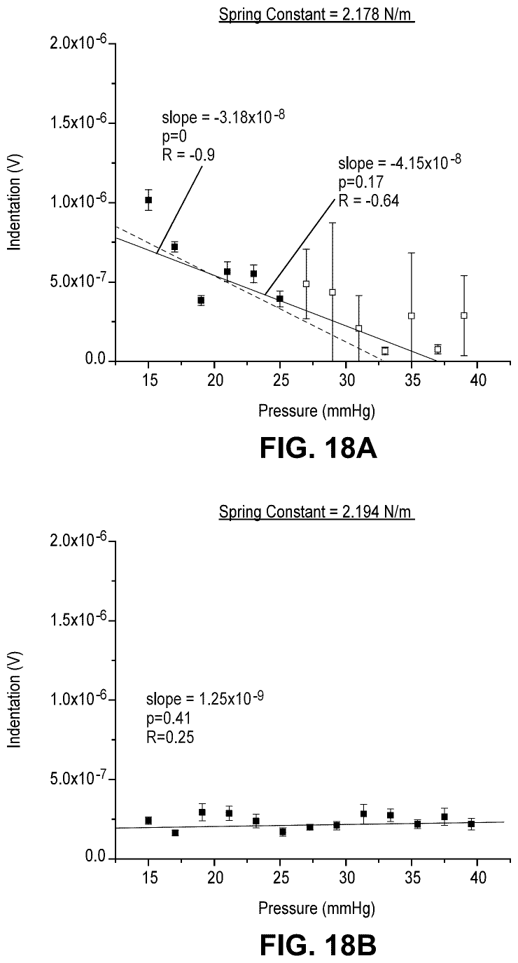

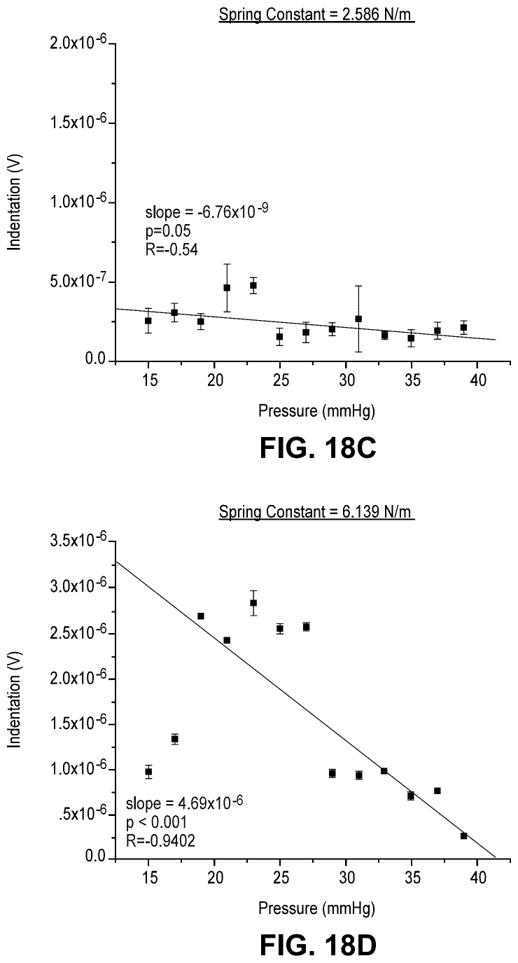

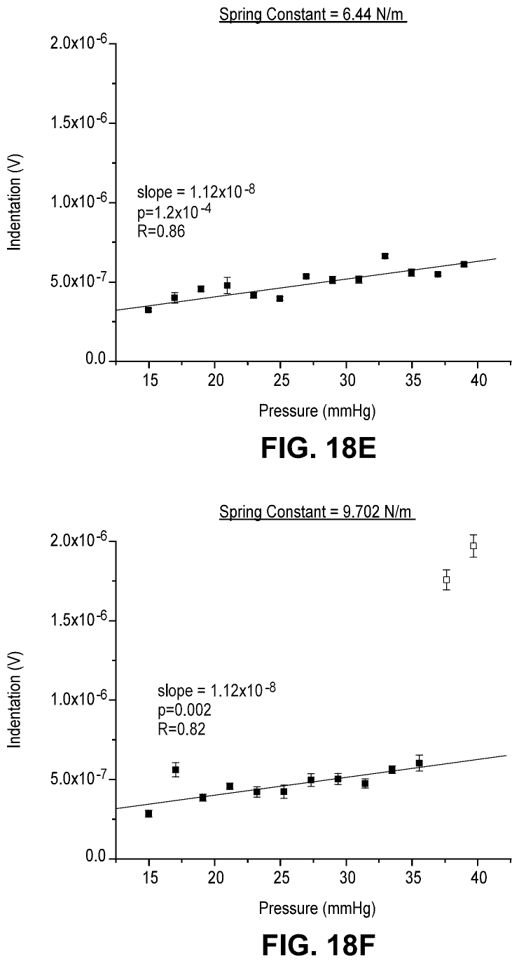

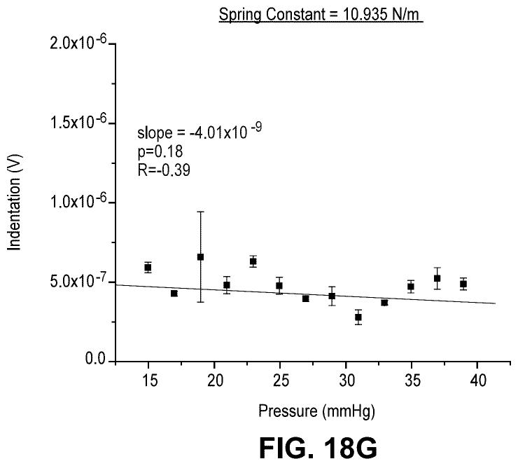

For each cantilever, the displacement data was graphed as a function of IOP. FIGS. 18A-18G are exemplary resultant data plots, showing the displacement (indentation) versus pressure (IOP) data corresponding to varying spring constants of the cantilevers used. A linear fit was applied to all the graphs, and the slope was determined. Based on the range of the data, a slope smaller than 5×10⁻⁹ V/mmHg was determined to be insignificant and within the noise level. Using this criterium, a linear trend between AFM measurements and IOP was found for 5 of the 13 cantilevers tested. The data shows that cantilevers with a spring constant lower than 6.44 N/m did not consistently output measurable differences with variations in IOP. In contrast, stiffer cantilevers produce a correlation more often and more reliably. Repeat measurements using stiffer cantilevers confirmed this trend.

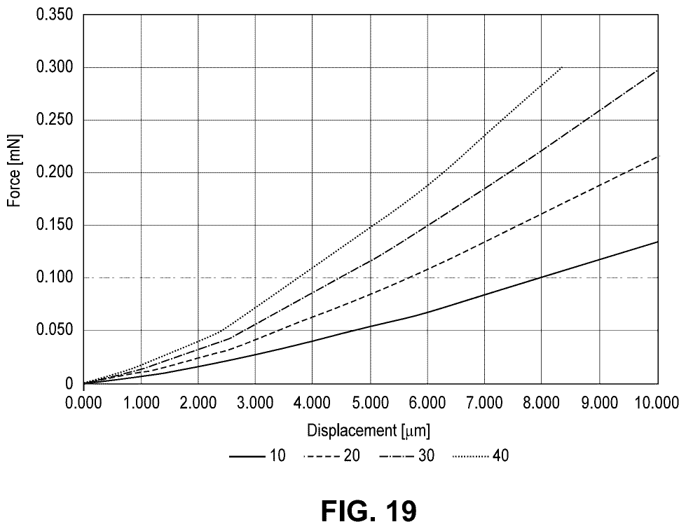

FIG. 19 is a plot showing the relationship between indentation force (vertical axis) as a function of indentation displacement or depth (horizontal axis), plotted for four different values of IOP ranging from 10 mmHg to 40 mmHg. Thus, in some embodiments, a change in the indentation force applied (and a corresponding change in resulting indentation depth) may be used as a way to fine tune (e.g., improve the sensitivity of) the estimation or assessment of IOP. This may be useful in cases where the IOP for a given patient has changed since the last measurement/assessment, for example.

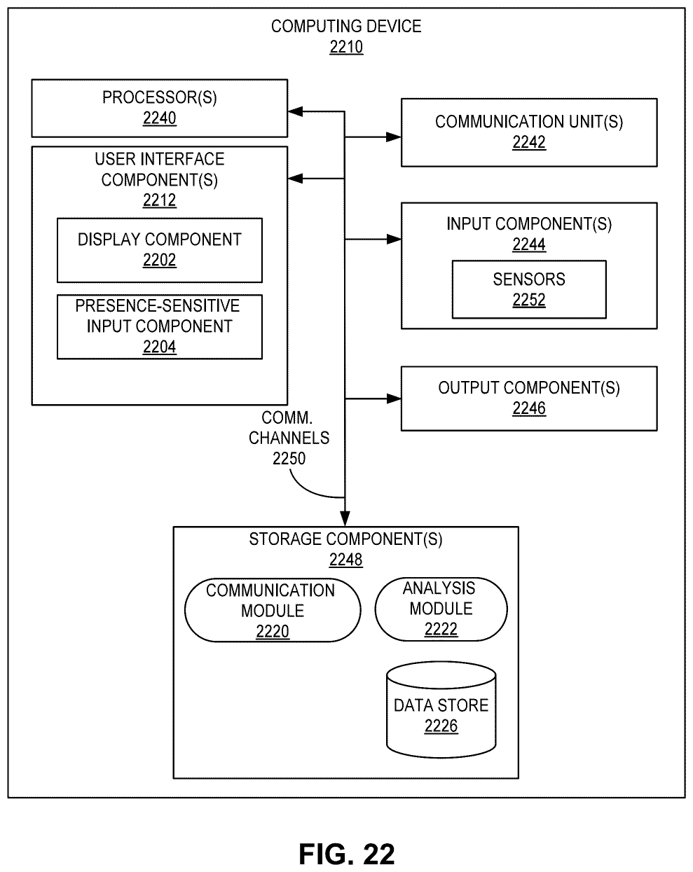

FIG. 22 is a block diagram illustrating a more detailed example of a computing device configured to perform the techniques described herein. FIG. 22 illustrates only one particular example of computing device 2210, and many other examples of computing device 2210 may be used in other instances and may include a subset of the components included in example computing device 2210 or may include additional components not shown in FIG. 22.

Computing device 2210 may be any computer with the processing power required to adequately execute the techniques described herein. For instance, computing device 2210 may be any one or more of a mobile computing device (e.g., a smartphone, a tablet computer, a laptop computer, etc.), a desktop computer, an integrated computer system, a wearable computing device (e.g., a smart watch, computerized glasses, a heart monitor, a glucose monitor, etc.), a network modem, router, or server system, or any other computerized device that may be configured to perform the techniques described herein.

As shown in the example of FIG. 22, computing device 2210 includes user interface components (UIC) 2212, one or more processors 2240, one or more communication units 2242, one or more input components 2244, one or more output components 2246, and one or more storage components 2248. UIC 2212 includes display component 2202 and presence-sensitive input component 2204. Storage components 2248 of computing device 2210 include communication module 2220, analysis module 2222, and data store 2226.

One or more processors 2240 may implement functionality and/or execute instructions associated with computing device 2210 to communicate with the contact lens system described herein. That is, processors 2240 may implement functionality and/or execute instructions associated with computing device 2210 to receive data from and send instructions to the contact lens system described herein to control said contact lens system wirelessly.

Examples of processors 2240 include any combination of application processors, display controllers, auxiliary processors, one or more sensor hubs, and any other hardware configured to function as a processor, a processing unit, or a processing device, including dedicated graphical processing units (GPUs). Modules 2220 and 2222 may be operable by processors 2240 to perform various actions, operations, or functions of computing device 2210. For example, processors 2240 of computing device 2210 may retrieve and execute instructions stored by storage components 2248 that cause processors 2240 to perform the operations described with respect to modules 2220 and 2222. The instructions, when executed by processors 2240, may cause computing device 2210 to receive data from and send instructions to the contact lens system described herein to control said contact lens system wirelessly.

Communication module 2220 may execute locally (e.g., at processors 2240) to provide functions associated with managing communications with the contact lens system and outputting various user interfaces (e.g., patient user interfaces or physician user interfaces). In some examples, communication module 2220 may act as an interface to a remote service accessible to computing device 2210. For example, communication module 2220 may be an interface or application programming interface (API) to a remote server that manages communications with the contact lens system and outputs various user interfaces (e.g., patient user interfaces or physician user interfaces).

In some examples, analysis module 2222 may execute locally (e.g., at processors 2240) to provide functions associated with comparing measured elasticity and IOP values to thresholds and determining updated therapies for the contact lens system to implement. For example, analysis module 2222 may be an interface or application programming interface (API) to a remote server that compares measured elasticity and IOP values to thresholds and determines updated therapies for the contact lens system to implement.

One or more storage components 2248 within computing device 2210 may store information for processing during operation of computing device 2210 (e.g., computing device 2210 may store data accessed by modules 2220 and 2222 during execution at computing device 2210). In some examples, storage component 2248 is a temporary memory, meaning that a primary purpose of storage component 2248 is not long-term storage. Storage components 2248 on computing device 2210 may be configured for short-term storage of information as volatile memory and therefore not retain stored contents if powered off. Examples of volatile memories include random access memories (RAM), dynamic random access memories (DRAM), static random access memories (SRAM), and other forms of volatile memories known in the art.

Storage components 2248, in some examples, also include one or more computer-readable storage media. Storage components 2248 in some examples include one or more non-transitory computer-readable storage mediums. Storage components 2248 may be configured to store larger amounts of information than typically stored by volatile memory. Storage components 2248 may further be configured for long-term storage of information as non-volatile memory space and retain information after power on/off cycles. Examples of non-volatile memories include magnetic hard discs, optical discs, floppy discs, flash memories, or forms of electrically programmable memories (EPROM) or electrically erasable and programmable (EEPROM) memories. Storage components 2248 may store program instructions and/or information (e.g., data) associated with modules 2220 and 2222 and data store 2226. Storage components 2248 may include a memory configured to store data or other information associated with modules 2220 and 2222 and data store 2226.

Communication channels 2250 may interconnect each of the components 2212, 2240, 2242, 2244, 2246, and 2248 for inter-component communications (physically, communicatively, and/or operatively). In some examples, communication channels 2250 may include a system bus, a network connection, an inter-process communication data structure, or any other method for communicating data.

One or more communication units 2242 of computing device 2210 may communicate with external devices via one or more wired and/or wireless networks by transmitting and/or receiving network signals on one or more networks. Examples of communication units 2242 include a network interface card (e.g., such as an Ethernet card), an optical transceiver, a radio frequency transceiver, a GPS receiver, a radio-frequency identification (RFID) transceiver, a near-field communication (NFC) transceiver, or any other type of device that can send and/or receive information. Other examples of communication units 2242 may include short wave radios, cellular data radios, wireless network radios, as well as universal serial bus (USB) controllers.

One or more input components 2244 of computing device 2210 may receive input. Examples of input are tactile, audio, and video input. Input components 2244 of computing device 2210, in one example, include a presence-sensitive input device (e.g., a touch sensitive screen, a PSD), mouse, keyboard, voice responsive system, camera, microphone or any other type of device for detecting input from a human or machine. In some examples, input components 2244 may include one or more sensor components (e.g., sensors 2252). Sensors 2252 may include one or more biometric sensors (e.g., fingerprint sensors, retina scanners, vocal input sensors/microphones, facial recognition sensors, cameras), one or more location sensors (e.g., GPS components, Wi-Fi components, cellular components), one or more temperature sensors, one or more movement sensors (e.g., accelerometers, gyros), one or more pressure sensors (e.g., barometer), one or more ambient light sensors, and one or more other sensors (e.g., infrared proximity sensor, hygrometer sensor, and the like). Other sensors, to name a few other non-limiting examples, may include a radar sensor, a lidar sensor, a sonar sensor, a heart rate sensor, magnetometer, glucose sensor, olfactory sensor, compass sensor, or a step counter sensor.

One or more output components 2246 of computing device 2210 may generate output in a selected modality. Examples of modalities may include a tactile notification, audible notification, visual notification, machine generated voice notification, or other modalities. Output components 2246 of computing device 2210, in one example, include a presence-sensitive display, a sound card, a video graphics adapter card, a speaker, a cathode ray tube (CRT) monitor, a liquid crystal display (LCD), a light emitting diode (LED) display, an organic LED (OLED) display, a three-dimensional display, or any other type of device for generating output to a human or machine in a selected modality.

UIC 2212 of computing device 2210 may include display component 2202 and presence-sensitive input component 2204. Display component 2202 may be a screen, such as any of the displays or systems described with respect to output components 2246, at which information (e.g., a visual indication) is displayed by UIC 2212 while presence-sensitive input component 2204 may detect an object at and/or near display component 2202.

While illustrated as an internal component of computing device 2210, UIC 2212 may also represent an external component that shares a data path with computing device 2210 for transmitting and/or receiving input and output. For instance, in one example, UIC 2212 represents a built-in component of computing device 2210 located within and physically connected to the external packaging of computing device 2210 (e.g., a screen on a mobile phone). In another example, UIC 2212 represents an external component of computing device 2210 located outside and physically separated from the packaging or housing of computing device 2210 (e.g., a monitor, a projector, etc. that shares a wired and/or wireless data path with computing device 2210).

UIC 2212 of computing device 2210 may detect two-dimensional and/or three-dimensional gestures as input from a user of computing device 2210. For instance, a sensor of UIC 2212 may detect a user's movement (e.g., moving a hand, an arm, a pen, a stylus, a tactile object, etc.) within a threshold distance of the sensor of UIC 2212. UIC 2212 may determine a two or three-dimensional vector representation of the movement and correlate the vector representation to a gesture input (e.g., a hand-wave, a pinch, a clap, a pen stroke, etc.) that has multiple dimensions. In other words, UIC 2212 can detect a multi-dimension gesture without requiring the user to gesture at or near a screen or surface at which UIC 2212 outputs information for display. Instead, UIC 2212 can detect a multi-dimensional gesture performed at or near a sensor which may or may not be located near the screen or surface at which UIC 2212 outputs information for display.

In accordance with the techniques of this disclosure, communication module 2220 may communicate with the contact lens system described throughout this disclosure, including the contact lens systems of FIGS. 5A-7, 9-11C, and 14A-14C. In some instances, communication module 2220 may send activation signals to the contact lens system, thereby causing the contact lens system to take measurements of elasticity of a patient's cornea or IOP of a patient's eye. Communication module 2220 may further receive a measured elasticity value and/or a measured IOP value from the contact lens system. Communication module 2220 may communicate with the contact lens system over a variety of methods dependent on the communication units present in the contact lens system, those methods including via server, via a Bluetooth® or other direct radio communication signal, via Near-Field Communication, or any other communication method suitable for incorporation into a contact lens system.

Computing device 2210 may either be a device operated by a patient or a device operated by a physician. In the instances where computing device 2210 is a patient computing device, analysis module 2222 may compare the measured elasticity value and/or the measured IOP value to a threshold. In response to the measured elasticity value and/or the measured IOP value exceeding the threshold, analysis module 2222 may generate a graphical user interface comprising a patient interface configured to take one or more corrective actions. The one or more corrective actions may include any one or more of contacting a physician, changing a therapy, or making no changes to the therapy. Changing the therapy may include any one or more of the following exemplary, non-limiting examples: changing which drug or medicated solution is to be delivered or eluted to the corneal surface of the patient's eye, changing an amount of the drug or drugs or medicated solutions to be delivered, and changing the frequency of delivery of drugs or medicated solutions, modifying or changing the composition or relative proportions of a mixture of drugs or medicated solutions to be delivered.

Communication module 2220 may output the graphical user interface via UIC 212 or some other output component 2246. In some examples, communication module 2220 may receive an indication of user input to change the therapy to a secondary therapy, and communication module 2220 may proceed to send instructions to the contact lens system to implement the secondary therapy. In other examples, analysis module 2222 may automatically determine the secondary therapy based on the measured elasticity value and/or the measured IOP value, and communication module 2220 may proceed to send instructions to the contact lens system to implement the automatically determined secondary therapy.

In other instances where computing device 2210 is a physician computing device, analysis module 2222 may generate a graphical user interface comprising a graphical indication of the measured elasticity value and/or the measured IOP value, as well as a physician interface configured to request input for performing any number of corrective actions. The one or more corrective actions may include any one or more of contacting a physician, changing a therapy, or making no changes to the therapy. Changing the therapy may include any one or more of the following exemplary, non-limiting examples: changing which drug or medicated solution is to be delivered or eluted to the corneal surface of the patient's eye, changing an amount of the drug or drugs or medicated solutions to be delivered, and changing the frequency of delivery of drugs or medicated solutions, modifying or changing the composition or relative proportions of a mixture of drugs or medicated solutions to be delivered.

Communication module 2220 may output the graphical user interface via UIC 212 or some other output component 2246. In some examples, communication module 2220 may receive an indication of user input to change the therapy to a secondary therapy, and communication module 2220 may proceed to send instructions to the contact lens system to implement the secondary therapy. In other examples, analysis module 2222 may automatically determine the secondary therapy based on the measured elasticity value and/or the measured IOP value, and communication module 2220 may proceed to send instructions to the contact lens system to implement the automatically determined secondary therapy.

By utilizing the techniques described herein, a natural interface for controlling the contact lens system described herein is provided. Naturally, by being located on a device the size of a contact lens, and by being placed on the surface of an eye of a human, the contact lens system described herein may not be capable of having an integrated user interface for making alterations to therapies performed by the contact lens system. While, in some instances, the therapy may be hard coded into the contact lens system, providing a user interface on a secondary computing device in communication with the contact lens system may provide additional benefits beyond the benefits realized by the contact lens system alone. As with any medical device, utilization of the medical device in an optimal way is generally inconsistent between patients, as each patient has their own physiological and medical differences that limit the benefits of singular approaches. By providing these user interfaces, both patients and physicians can control the treatment or prophylaxis of the contact lens system in an individualistic manner to optimally perform for the individual patient wearing the contact lens system.

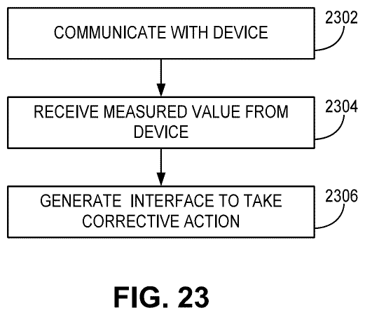

FIG. 23 is a flow diagram illustrating an example process for generating a patient interface, in accordance with one or more techniques of this disclosure. The techniques of FIG. 23 may be performed by one or more processors of a computing device, such as computing device 2210 illustrated in FIG. 22. For purposes of illustration only, the techniques of FIG. 23 are described within the context of computing device 2210 of FIG. 22, although computing devices having configurations different than that of computing device 2210 may perform the techniques of FIG. 23.

In accordance with the techniques of this disclosure, communication module 2220 communicates with a device, such as a contact lens system described herein (2302). Communication module 2220 receives a measured value from the device, such as a measured elasticity value or a measured IOP value (2304). Analysis module 2222 generates an interface to take a corrective action, such as when the measured value exceeds a threshold (2306).

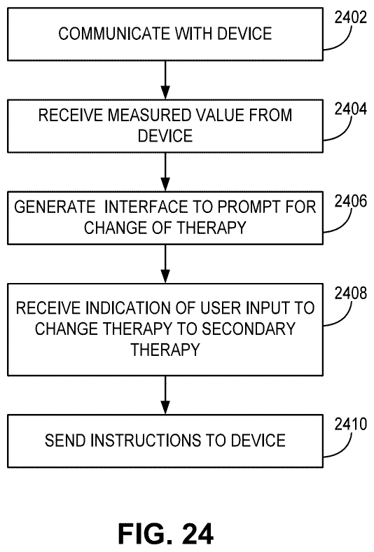

FIG. 24 is a flow diagram illustrating an example process for generating a physician interface, in accordance with one or more techniques of this disclosure. The techniques of FIG. 24 may be performed by one or more processors of a computing device, such as computing device 2210 illustrated in FIG. 22. For purposes of illustration only, the techniques of FIG. 24 are described within the context of computing device 2210 of FIG. 22, although computing devices having configurations different than that of computing device 2210 may perform the techniques of FIG. 24.

In accordance with the techniques of this disclosure, communication module 2220 communicates with a device, such as a contact lens system described herein (2402). Communication module 2220 receives a measured value from the device, such as a measured elasticity value or a measured IOP value (2404). Analysis module 2222 generates an interface to prompt a physician for a change of therapy for the patient associated with the measured value (2406). Communication module 2220 receives an indication of user input to change the therapy to a secondary therapy (2408). Communication module 2220 sends instructions to the device to implement the secondary therapy at the device (2410).

While the various systems described above are separate implementations, any of the individual components, mechanisms, or devices, and related features and functionality, within the various system embodiments described in detail above can be incorporated into any of the other system embodiments herein.

The terms “about” and “substantially,” as used herein, refers to variation that can occur (including in numerical quantity or structure), for example, through typical measuring techniques and equipment, with respect to any quantifiable variable, including, but not limited to, mass, volume, time, distance, wavelength, frequency, voltage, current, and electromagnetic field. Further, there is certain inadvertent error and variation in the real world that is likely through differences in the manufacture, source, or precision of the components used to make the various components or carry out the methods and the like. The terms “about” and “substantially” also encompass these variations. The term “about” and “substantially” can include any variation of 5% or 10%, or any amount-including any integer-between 0% and 10%. Further, whether or not modified by the term “about” or “substantially,” the claims include equivalents to the quantities or amounts.

Numeric ranges recited within the specification are inclusive of the numbers defining the range and include each integer within the defined range. Throughout this disclosure, various aspects of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 11/2, and 43/4 This applies regardless of the breadth of the range. Although the various embodiments have been described with reference to preferred implementations, persons skilled in the art will recognize that changes may be made in form and detail without departing from the spirit and scope thereof.

It is to be recognized that depending on the example, certain acts or events of any of the techniques described herein can be performed in a different sequence, may be added, merged, or left out altogether (e.g., not all described acts or events are necessary for the practice of the techniques). Moreover, in certain examples, acts or events may be performed concurrently, e.g., through multi-threaded processing, interrupt processing, or multiple processors, rather than sequentially.

In one or more examples, the functions described may be implemented in hardware, software, firmware, or any combination thereof. If implemented in software, the functions may be stored on or transmitted over as one or more instructions or code on a computer-readable medium and executed by a hardware-based processing unit. Computer-readable media may include computer-readable storage media, which corresponds to a tangible medium such as data storage media, or communication media including any medium that facilitates transfer of a computer program from one place to another, e.g., according to a communication protocol. In this manner, computer-readable media generally may correspond to (1) tangible computer-readable storage media which is non-transitory or (2) a communication medium such as a signal or carrier wave. Data storage media may be any available media that can be accessed by one or more computers or one or more processors to retrieve instructions, code and/or data structures for implementation of the techniques described in this disclosure. A computer program product may include a computer-readable medium.

It is contemplated that the various aspects, features, processes, and operations from the various embodiments may be used in any of the other embodiments unless expressly stated to the contrary. Certain operations illustrated may be implemented by a computer executing a computer program product on a non-transient, computer-readable storage medium, where the computer program product includes instructions causing the computer to execute one or more of the operations, or to issue commands to other devices to execute one or more operations.

By way of example, and not limitation, such computer-readable storage media can comprise RAM, ROM, EEPROM, CD-ROM or other optical disk storage, magnetic disk storage, or other magnetic storage devices, flash memory, or any other medium that can be used to store desired program code in the form of instructions or data structures and that can be accessed by a computer. Also, any connection is properly termed a computer-readable medium. For example, if instructions are transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. It should be understood, however, that computer-readable storage media and data storage media do not include connections, carrier waves, signals, or other transitory media, but are instead directed to non-transitory, tangible storage media. Disk and disc, as used herein, includes compact disc (CD), laser disc, optical disc, digital versatile disc (DVD), floppy disk and Blu-ray disc, where disks usually reproduce data magnetically, while discs reproduce data optically with lasers. Combinations of the above should also be included within the scope of computer-readable media.

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor,” as used herein may refer to any of the foregoing structure or any other structure suitable for implementation of the techniques described herein. In addition, in some aspects, the functionality described herein may be provided within dedicated hardware and/or software modules configured for encoding and decoding, or incorporated in a combined codec. Also, the techniques could be fully implemented in one or more circuits or logic elements.

The techniques of this disclosure may be implemented in a wide variety of devices or apparatuses, including a wireless handset, an integrated circuit (IC) or a set of ICs (e.g., a chip set). Various components, modules, or units are described in this disclosure to emphasize functional aspects of devices configured to perform the disclosed techniques, but do not necessarily require realization by different hardware units. Rather, as described above, various units may be combined in a codec hardware unit or provided by a collection of interoperative hardware units, including one or more processors as described above, in conjunction with suitable software and/or firmware.

Various embodiments of the invention may be implemented at least in part in any conventional computer programming language. For example, some embodiments may be implemented in a procedural programming language (e.g., “C”), or in an object oriented programming language (e.g., “C++”). Other embodiments of the invention may be implemented as a pre-configured, stand-alone hardware element and/or as preprogrammed hardware elements (e.g., application specific integrated circuits, FPGAs, and digital signal processors), or other related components.

Those skilled in the art should appreciate that such computer instructions can be written in a number of programming languages for use with many computer architectures or operating systems. Furthermore, such instructions may be stored in any memory device, such as semiconductor, magnetic, optical or other memory devices, and may be transmitted using any communications technology, such as optical, infrared, microwave, or other transmission technologies.

Among other ways, such a computer program product may be distributed as a removable medium with accompanying printed or electronic documentation (e.g., shrink wrapped software), preloaded with a computer system (e.g., on system ROM or fixed disk), or distributed from a server or electronic bulletin board over the network (e.g., the Internet or World Wide Web). In fact, some embodiments may be implemented in a software-as-a-service model (“SAAS”) or cloud computing model. Of course, some embodiments of the invention may be implemented as a combination of both software (e.g., a computer program product) and hardware. Still other embodiments of the invention are implemented as entirely hardware, or entirely software.

Although the various embodiments have been described with reference to preferred implementations, persons skilled in the art will recognize that changes may be made in form and detail without departing from the spirit and scope thereof.

What is claimed is:

1. A device for assessing intra-ocular pressure (IOP) of a patient's eye, the device comprising:
(a) a wearable contact lens; and
(b) at least one cantilever sensor mounted to the contact lens, the cantilever sensor configured to measure an elasticity of a cornea of the patient's eye and/or an IOP of the patient's eye;
wherein the contact lens is configured to position the at least one cantilever sensor adjacent to a surface of the cornea of the patient's eye, and
wherein the cantilever sensor comprises a cantilever tip configured to apply a force to the surface of the cornea.

2. The device of claim 1, wherein the cantilever sensor is curved to conform to the surface of the cornea.

3. The device of claim 1, wherein the device comprises a plurality of cantilever sensors disposed outside a central optical zone of the contact lens.

4. The device of claim 3, wherein the device wherein the plurality of cantilever sensors is disposed in a paracentral zone of the contact lens.

5. The device of claim 3, wherein the device is configured to compute an average elasticity of the cornea and/or an average IOP of the patient's eye based on the elasticity and/or IOP measured by the plurality of cantilever sensors disposed outside the central optical zone of the contact lens.

6. The device of claim 5, wherein the device further comprises a processor configured to compute the average elasticity and/or the average IOP.

7. The device of claim 5, wherein the device further comprises an antenna configured to transmit data regarding the average elasticity and/or the average IOP to an external device.

8. The device of claim 1, wherein the contact lens is a corneal contact lens.

9. The device of claim 8, wherein the corneal contact lens has a diameter of less than 14.5 mm.

10. The device of claim 1, wherein the contact lens is a scleral contact lens.

11. The device of claim 10, wherein the scleral contact lens comprises an outer scleral skirt formed of a first material that is different from a second material forming an inner portion of the scleral contact lens.

12. The device of claim 1, wherein the contact lens is a hybrid contact lens.

13. The device of claim 1, wherein the device further comprises an antenna coupled to the at least one cantilever sensor, the antenna configured to transmit data regarding measured elasticity and/or IOP to an external device.

14. The device of claim 13, wherein the antenna is configured to transmit data to the external device wirelessly.

15. The device of claim 14, wherein the external device is configured to upload the transmitted data to a cloud database.

16. The device of claim 1, wherein the device further comprises a drug within the contact lens, the drug configured to be eluted from the contact lens to the patient.

17. The device of claim 16 wherein the drug is eluted to the patient's eye based upon a measured elasticity value and/or IOP value exceeding a threshold.

18. The device of claim 1 further configured to be calibrated to determine IOP based on one or more patient specific parameters.

19. The device of claim 18, wherein the device is calibrated to determine IOP based on a measured corneal thickness of the patient's eye.

20. The device of claim 18, wherein the device is calibrated to determine IOP based on a measured corneal radius of curvature of the patient's eye.

21. The device of claim 1, wherein the wearable contact lens further comprises at least one application-specific integrated circuit (“ASIC”) operably coupled to the at least one cantilever sensor, the ASIC configured to perform one or more of the following:
control actuation of the at least one cantilever sensor;
control data flow to and from the at least one cantilever sensor;
receive electrical power from a battery or a capacitor; and
communicate with an external data via an antenna disposed on the wearable contact lens.

22. A software application for use with a device for assessing intra-ocular pressure of a patient's eye, the device comprising:
(a) a wearable contact lens; and
(b) at least one cantilever sensor mounted to the contact lens, the cantilever sensor configured to measure an elasticity of a cornea of the patient's eye and/or an IOP of the patient's eye,
wherein the contact lens is configured to position the at least one cantilever sensor adjacent to a surface of the cornea of the patient's eye, and
wherein the cantilever sensor comprises a cantilever tip configured to apply a force to the surface of the cornea,
the software application including a patient user interface configured to cause one or more of the following actions to be performed in response to the measured elasticity value and/or the IOP value exceeding a threshold: (a) contact a physician, (b) recommend changing a therapy, or (c) make no changes.

23. A software application for use with a device for assessing intra-ocular pressure of a patient's eye the device comprising:
(a) a wearable contact lens; and
(b) at least one cantilever sensor mounted to the contact lens, the cantilever sensor configured to measure an elasticity of a cornea of the patient's eye and/or an IOP of the patient's eye,
wherein the contact lens is configured to position the at least one cantilever sensor adjacent to a surface of the cornea of the patient's eye, and
wherein the cantilever sensor comprises a cantilever tip configured to apply a force to the surface of the cornea,
the software application including a physician user interface configured to cause one or more of the following actions to be performed: (a) contact a patient, (b) recommend a change a therapy, or (c) make no changes.

24. A non-transitory computer-readable storage medium having stored thereon instructions that, when executed, cause one or more processors of a computing device to:
communicate with the device of claim 1;
receive a measured elasticity value and/or a measured IOP value from the device of claim 1;
in response to the measured elasticity value and/or the measured IOP value exceeding a threshold, generate a graphical user interface comprising a patient interface configured to take one or more corrective actions, the one or more corrective actions comprising:
(a) contact a physician,
(b) recommend a change in a therapy, or
(c) make no changes.

25. A non-transitory computer-readable storage medium having stored thereon instructions that, when executed, cause one or more processors of a computing device to:
communicate with the device of claim 1;
receive a measured elasticity value and/or a measured IOP value from the device of claim 1;
generate a graphical user interface comprising a graphical indication of the measured elasticity value and/or the measured IOP value and a physician interface configured to do one or more of the following:
(a) contact a patient,
(b) recommend a change in a therapy, or
(c) make no changes.

26. The non-transitory computer-readable storage medium of claim 25, wherein the physician interface is configured to change the therapy, and wherein the instructions, when executed, cause the one or more processors to:
receive an indication of user input to change the therapy to a secondary therapy; and
send instructions to the device of claim 1 to implement the secondary therapy.

27. A storage device for storing the device of claim 1, the storage device comprising a housing for holding the device, the housing configured to perform one or more of the following functions:
store the device in a cleaning solution to clean the wearable contact lens and the at least one cantilever sensor of the device;
store the device in a medicated solution to load the wearable contact lens of the device with the medicated solution while the device is stored in the housing;
deliver electrical power to charge a power supply and/or a battery of the device while the device is stored in the housing;
facilitate transmission of data from the device to a storage device; and
facilitate transmission of data and/or instructions to the device from an external device.
